SEQUENCE LISTING (116 sequences)

SEQ ID NO: 1 - protein, 20 aa, Human papillomavirus
MSLWLPSEATVYLPPVPVSK

SEQ ID NO: 2 - protein, 20 aa, Human papillomavirus
YLPPVPVSKVVSTDEYVART

SEQ ID NO: 3 - protein, 20 aa, Human papillomavirus
STDEYVARTNIYYHAGTSRL

SEQ ID NO: 4 - protein, 20 aa, Human papillomavirus
YYHAGTSRLLAVGHPYFPIK

SEQ ID NO: 5 - protein, 20 aa, Human papillomavirus
VGHPYFPIKKPNNNKILVPK

SEQ ID NO: 6 - protein, 20 aa, Human papillomavirus
NNNKILVPKVSGLQYRVFRI

SEQ ID NO: 7 - protein, 20 aa, Human papillomavirus
GLQYRVFRIHLPDPNKFGFP

SEQ ID NO: 8 - protein, 20 aa, Human papillomavirus
PDPNKFGFPDTSFYNPDTQR

SEQ ID NO: 9 - protein, 20 aa, Human papillomavirus
SFYNPDTQRLVWACVGVEVG

SEQ ID NO: 10 - protein, 20 aa, Human papillomavirus
WACVGVEVGRGQPLGVGISG

SEQ ID NO: 11 - protein, 20 aa, Human papillomavirus
QPLGVGISGHPLLNKLDDTE

SEQ ID NO: 12 - protein, 20 aa, Human papillomavirus
LLNKLDDTENASAYAANAGV

SEQ ID NO: 13 - protein, 20 aa, Human papillomavirus
SAYAANAGVDNRECISMDYK

SEQ ID NO: 14 - protein, 20 aa, Human papillomavirus
RECISMDYKQTQLCLIGCKP

SEQ ID NO: 15 - protein, 20 aa, Human papillomavirus
QLCLIGCKPPIGEHWGKGSP

SEQ ID NO: 16 - protein, 20 aa, Human papillomavirus
GEHWGKGSPCTNVAVNPGDC

SEQ ID NO: 17 - protein, 20 aa, Human papillomavirus
NVAVNPGDCPPLELINTVIQ

SEQ ID NO: 18 - protein, 20 aa, Human papillomavirus
LELINTVIQDGDMVDTGFGA

SEQ ID NO: 19 - protein, 20 aa, Human papillomavirus
DMVDTGFGAMDFTTLQANKS

SEQ ID NO: 20 - protein, 20 aa, Human papillomavirus
FTTLQANKSEVPLDICTSIC

SEQ ID NO: 21 - protein, 20 aa, Human papillomavirus
PLDICTSICKYPDYIKMVSE

SEQ ID NO: 22 - protein, 20 aa, Human papillomavirus
PDYIKMVSEPYGDSLFFYLR

SEQ ID NO: 23 - protein, 20 aa, Human papillomavirus
GDSLFFYLRREQMFVRHLFN

SEQ ID NO: 24 - protein, 20 aa, Human papillomavirus
QMFVRHLFNRAGAVGENVPD

SEQ ID NO: 25 - protein, 20 aa, Human papillomavirus
GAVGENVPDDLYIKGSGSTA

SEQ ID NO: 26 - protein, 20 aa, Human papillomavirus
YIKGSGSTANLASSNYFPTP

SEQ ID NO: 27 - protein, 20 aa, Human papillomavirus
ASSNYFPTPSGSMVTSDAQI

SEQ ID NO: 28 - protein, 20 aa, Human papillomavirus
SMVTSDAQIFNKPYWLQRAQ

SEQ ID NO: 29 - protein, 20 aa, Human papillomavirus
KPYWLQRAQGHNNGICWGNQ

SEQ ID NO: 30 - protein, 20 aa, Human papillomavirus
NNGICWGNQLFVTVVDTTRS

SEQ ID NO: 31 - protein, 20 aa, Human papillomavirus
VTVVDTTRSTNMSLCAAIST

SEQ ID NO: 32 - protein, 20 aa, Human papillomavirus
MSLCAAISTSETTYKNTNFK

SEQ ID NO: 33 - protein, 20 aa, Human papillomavirus
TTYKNTNFKEYLRHGEEYDL

SEQ ID NO: 34 - protein, 20 aa, Human papillomavirus
LRHGEEYDLQFIFQLCKITL

SEQ ID NO: 35 - protein, 20 aa, Human papillomavirus
IFQLCKITLTADVMTYIHSM

SEQ ID NO: 36 - protein, 20 aa, Human papillomavirus
DVMTYIHSMNSTILEDWNFG

SEQ ID NO: 37 - protein, 20 aa, Human papillomavirus
TILEDWNFGLQPPPGGTLED

SEQ ID NO: 38 - protein, 20 aa, Human papillomavirus
PPPGGTLEDTYRFVTSQAIA

SEQ ID NO: 39 - protein, 20 aa, Human papillomavirus
RFVTSQAIACQKHTPPAPKE

SEQ ID NO: 40 - protein, 20 aa, Human papillomavirus
KHTPPAPKEDPLKKYTFWEV

SEQ ID NO: 41 - protein, 20 aa, Human papillomavirus
LKKYTFWEVNLKEKFSADLD

SEQ ID NO: 42 - protein, 20 aa, Human papillomavirus
KEKFSADLDQFPLGRKFLLQ

SEQ ID NO: 43 - protein, 20 aa, Human papillomavirus
PLGRKFLLQAGMHGDTPTLH

SEQ ID NO: 44 - protein, 20 aa, Human papillomavirus
MHGDTPTLHEYMLDLQPETT

SEQ ID NO: 45 - protein, 20 aa, Human papillomavirus
MLDLQPETTDLYCYEQLNDS

SEQ ID NO: 46 - protein, 20 aa, Human papillomavirus
YCYEQLNDSSEEEDEIDGPA

SEQ ID NO: 47 - protein, 20 aa, Human papillomavirus
EEDEIDGPAGQAEPDRAHYN

SEQ ID NO: 48 - protein, 20 aa, Human papillomavirus
AEPDRAHYNIVTFCCKCDST

SEQ ID NO: 49 - protein, 20 aa, Human papillomavirus
TFCCKCDSTLRLCVQSTHVD

SEQ ID NO: 50 - protein, 20 aa, Human papillomavirus
LCVQSTHVDIRTLEDLLMGT

SEQ ID NO: 51 - protein, 21 aa, Human papillomavirus
TLEDLLMGTLGIVCPICSQKP

SEQ ID NO: 52 - protein, 20 aa, Human papillomavirus
MALWRPSDNTVYLPPPSVAR

SEQ ID NO: 53 - protein, 20 aa, Human papillomavirus
YLPPPSVARVVNTDDYVTRT

SEQ ID NO: 54 - protein, 20 aa, Human papillomavirus
NTDDYVTRTSIFYHAGSSRL

SEQ ID NO: 55 - protein, 20 aa, Human papillomavirus
FYHAGSSRLLTVGNPYFRVP

SEQ ID NO: 56 - protein, 20 aa, Human papillomavirus
VGNPYFRVPAGGGNKQDIPK

SEQ ID NO: 57 - protein, 20 aa, Human papillomavirus
GGNKQDIPKVSAYQYRVFRV

SEQ ID NO: 58 - protein, 20 aa, Human papillomavirus
AYQYRVFRVQLPDPNKFGLP

SEQ ID NO: 59 - protein, 20 aa, Human papillomavirus
PDPNKFGLPDTSIYNPETQR

SEQ ID NO: 60 - protein, 20 aa, Human papillomavirus
SIYNPETQRLVWACAGVEIG

SEQ ID NO: 61 - protein, 20 aa, Human papillomavirus
WACAGVEIGRGQPLGVGLSG

SEQ ID NO: 62 - protein, 20 aa, Human papillomavirus
QPLGVGLSGHPFYNKLDDTE

SEQ ID NO: 63 - protein, 20 aa, Human papillomavirus
FYNKLDDTESSHAATSNVSE

SEQ ID NO: 64 - protein, 20 aa, Human papillomavirus
HAATSNVSEDVRDNVSVDYK

SEQ ID NO: 65 - protein, 20 aa, Human papillomavirus
RDNVSVDYKQTQLCILGCAP

SEQ ID NO: 66 - protein, 20 aa, Human papillomavirus
QLCILGCAPAIGEHWAKGTA

SEQ ID NO: 67 - protein, 20 aa, Human papillomavirus
GEHWAKGTACKSRPLSQGDC

SEQ ID NO: 68 - protein, 20 aa, Human papillomavirus
SRPLSQGDCPPLELKNTVLE

SEQ ID NO: 69 - protein, 20 aa, Human papillomavirus
LELKNTVLEDGDMVDTGYGA

SEQ ID NO: 70 - protein, 20 aa, Human papillomavirus
DMVDTGYGAMDFSTLQDTKC

SEQ ID NO: 71 - protein, 20 aa, Human papillomavirus
FSTLQDTKCEVPLDICQSIC

SEQ ID NO: 72 - protein, 20 aa, Human papillomavirus
PLDICQSICKYPDYLQMSAD

SEQ ID NO: 73 - protein, 20 aa, Human papillomavirus
PDYLQMSADPYGDSMFFCLR

SEQ ID NO: 74 - protein, 20 aa, Human papillomavirus
GDSMFFCLRREQLFARHFWN

SEQ ID NO: 75 - protein, 20 aa, Human papillomavirus
QLFARHFWNRAGTMGDTVPQ

SEQ ID NO: 76 - protein, 20 aa, Human papillomavirus
GTMGDTVPQSLYIKGTGMRA

SEQ ID NO: 77 - protein, 20 aa, Human papillomavirus
YIKGTGMRASPGSCVYSPSP

SEQ ID NO: 78 - protein, 20 aa, Human papillomavirus
GSCVYSPSPSGSIVTSDSQL

SEQ ID NO: 79 - protein, 20 aa, Human papillomavirus
SIVTSDSQLFNKPYWLHKAQ

SEQ ID NO: 80 - protein, 20 aa, Human papillomavirus
KPYWLHKAQGHNNGVCWHNQ

SEQ ID NO: 81 - protein, 20 aa, Human papillomavirus
NNGVCWHNQLFVTVVDTTRS

SEQ ID NO: 82 - protein, 20 aa, Human papillomavirus
VTVVDTTRSTNLTICASTQS

SEQ ID NO: 83 - protein, 20 aa, Human papillomavirus
LTICASTQSPVPGQYDATKF

SEQ ID NO: 84 - protein, 20 aa, Human papillomavirus
PGQYDATKFKQYSRHVEEYD

SEQ ID NO: 85 - protein, 20 aa, Human papillomavirus
YSRHVEEYDLQFIFQLCTIT

SEQ ID NO: 86 - protein, 20 aa, Human papillomavirus
FIFQLCTITLTADVMSYIHS

SEQ ID NO: 87 - protein, 20 aa, Human papillomavirus
ADVMSYIHSMNSSILEDWNF

SEQ ID NO: 88 - protein, 20 aa, Human papillomavirus
SSILEDWNFGVPPPPTTSLV

SEQ ID NO: 89 - protein, 20 aa, Human papillomavirus
PPPPTTSLVDTYRFVQSVAI

SEQ ID NO: 90 - protein, 20 aa, Human papillomavirus
YRFVQSVAITCQKDAAPAEN

SEQ ID NO: 91 - protein, 20 aa, Human papillomavirus
QKDAAPAENKDPYDKLKFWN

SEQ ID NO: 92 - protein, 20 aa, Human papillomavirus
PYDKLKFWNVDLKEKFSLDL

SEQ ID NO: 93 - protein, 20 aa, Human papillomavirus
LKEKFSLDLDQYPLGRKFLV

SEQ ID NO: 94 - protein, 20 aa, Human papillomavirus
YPLGRKFLVQAGMHGPKATL

SEQ ID NO: 95 - protein, 20 aa, Human papillomavirus
MHGPKATLQDIVLHLEPQNE

SEQ ID NO: 96 - protein, 20 aa, Human papillomavirus
VLHLEPQNEIPVDLLCHEQL

SEQ ID NO: 97 - protein, 20 aa, Human papillomavirus
VDLLCHEQLSDSEEENDEID

SEQ ID NO: 98 - protein, 20 aa, Human papillomavirus
SEEENDEIDGVNHQHLPARR

SEQ ID NO: 99 - protein, 20 aa, Human papillomavirus
NHQHLPARRAEPQRHTMLCM

SEQ ID NO: 100 - protein, 20 aa, Human papillomavirus
PQRHTMLCMCCKCEARIKLV

SEQ ID NO: 101 - protein, 20 aa, Human papillomavirus
KCEARIKLVVESSADDLRAF

SEQ ID NO: 102 - protein, 20 aa, Human papillomavirus
SSADDLRAFQQLFLNTLSFV

SEQ ID NO: 103 - protein, 17 aa, Human papillomavirus
LFLNTLSFVCPWCASQQ

SEQ ID NO: 104 - DNA, 36 bp, Human papillomavirus
accagactcgagatggctttgtggcggcctagtgac

SEQ ID NO: 105 - DNA, 42 bp, Human papillomavirus
atagccaagcttaatgatatcctgaaccaaaaatttacgtcc

SEQ ID NO: 106 - DNA, 36 bp, Human papillomavirus
ggccatgatatcatgcatggacctaaggcaacattg

SEQ ID NO: 107 - DNA, 35 bp, Human papillomavirus
ggccatgatatctcgtcgggctggtaaatgtgatg

SEQ ID NO: 108 - DNA, 35 bp, Human papillomavirus
ggccatgatatctgtgtgacgttgtggttcggctc

SEQ ID NO: 109 - protein, 20 aa, Human papillomavirus
NTDDYVTRTSIFYHAGSSRL

SEQ ID NO: 110 - protein, 20 aa, Human papillomavirus
FYHAGSSRLLTVGNPYFRVP

SEQ ID NO: 111 - protein, 20 aa, Human papillomavirus
PQRHTMLCMCCKCEARIKLV

SEQ ID NO: 112 - protein, 9 aa, Human papillomavirus
GMHGPKATL

SEQ ID NO: 113 - protein, 10 aa, Human papillomavirus
HGPKATLQDI

SEQ ID NO: 114 - protein, 8 aa, Human papillomavirus
MHGPKATL

SEQ ID NO: 115 - protein, 9 aa, Human papillomavirus
FQQLFLNTL

SEQ ID NO: 116 - protein, 9 aa, Human papillomavirus
IYNPETQRL

(19) United States
(12) Patent Application Publication
(10) Pub. No.: US 2004/0091479 A1
(43) Pub. Date: May 13, 2004

(54) T-cell epitope of the papillomavirus l1 and e7 protein and use thereof in diagnostics and therapy

(76) Inventors: John Nieland, Stockdorf (DE); Andreas Kaufmann, Jena (DE); Angela Kather, Jena (DE); Manuela Schinz, Ranis (DE)

Correspondence Address: CLARK & ELBING LLP, 101 FEDERAL STREET, BOSTON, MA, 02110, US

(21) Appl. No.: 10/432,465

(22) PCT Filed: Nov. 30, 2001
(86) PCT No.: PCT/EP01/14037

(30) Foreign Application Priority Data
Dec. 1, 2000 (DE) 10059631.2

(51) Int. Cl. C07H 21/04; A61K 39/395; C07K 14/74; C12N 5/08

(52) U.S. Cl. 424/144.1; 530/350; 435/69.1; 435/320.1; 435/372; 536/23.5

(57) ABSTRACT

The present invention relates to a papilloma virus T cell epitope having an amino acid sequence

YLPPVPVSKVVSTDEYVART, STDEYVARTNIYYHAGTSRL,
VGHPYFPIKKPNNNKILVPK, GLQYRVFRIHLPDPNKFGFP,
WACVGVEVGRGQPLGVGISG, QPLGVGISGHPLLNKLDDTE,
QLCLIGCKPPIGEHWGKGSP, LELINTVIQDGDMVDTGFGA,
DMVDTGFGANDFTTLQANKS, VTVVDTTRSTNNSLCAAIST,
TTYKNTNFKEYLRHGEEYDL, IFQLCKITLTADVMTYIHSM,
PPPGGTLEDTYRFVTSQAIA, RFVTSQAIACQKHTPPAPKB,
LKKYTFWEVNLKEKFSADLD, PLGRKFLLQAGMHGDTPTLH,
YCYEQLNDSSEEEDEIDGPA, VGNPYFRVPAGGGNKQDIPK,
GGNKQDIPKVSAYQYRVFRV, SIYNPETQRLVWACAGVEIG,
IYNPETQRL, PDYLQMSADPYGDSMFFCLR,
GDSMFFCLRREQLFARHFWN, NNGVCWHNQLFVTVVDTTRS,
PPPPTTSLVDTYRFVQSVAI, YRFVQSVAITCQKDAAPAEN,
PYDKLKFWNVDLKEKFSLDL, YPLGRKFLVQAGMHGPKATL,
MHGPKATLQDIVLHLEPQNE, VDLLCHEQLSDSEEENDEID,
SEEENDEIDGVNHQHLPARR, SSADDLPAFQQLFLNTLSFV
NTDDYVTRTSIFYHAGSSRL, FYHAGSSRLLTVGNPYFRVP,
PQRHTMLCMCCKCEARIKLV, GMHGPKATL, HGPKATLQDI,
MHGPKATL, or FQQLFLNTL

and/or a functionally active variant thereof, and to its use in diagnosis and therapy.

The present invention relates to a papillomavirus T cell epitope having an amino acid sequence

YLPPVPVSKVVSTDEYVART, STDEYVARTNIYYHAGTSRL, VGHPYFPIKKPNNNKILVPK,
GLQYRVFRIHLPDPNKFGFP, WACVGVEVGRGQPLGVGISG, QPLGVGISGHPLLNKLDDTE,
QLCLIGCKPPIGEHWGKGSP, LELINTVIQDGDMVDTGFGA, DMVDTGFGAMDFTTLQANKS,
VTVVDTTRSTNMSLCAAIST, TTYKNTNFKEYLRHGEEYDL, IFQLCKITLTADVMTYIHSM,
PPPGGTLEDTYRFVTSQAIA, RFVTSQAIACQKHTPPAPKE, LKKYTFWEVNLKEKFSADLD,
PLGRKFLLQAGMHGDTPTLH, YCYEQLNDSSEEEDEIDGPA, VGNPYFRVPAGGGNKQDIPK,
GGNKQDIPKVSAYQYRVFRV, SIYNPETQRLVWACAGVEIG, IYNPETQRL,
PDYLQMSADPYGDSMFFCLR, GDSMFFCLRREQLFARHFWN,
NNGVCWHNQLFVTVVDTTRS, PPPPTTSLVDTYRFVQSVAI, YRFVQSVAITCQKDAAPAEN,
PYDKLKFWNVDLKEKFSLDL, YPLGRKFLVQAGMHGPKATL, MHGPKATLQDIVLHLEPQNE,
VDLLCHEQLSDSEEENDEID, SEEENDEIDGVNHQHLPARR, SSADDLRAFQQLFLNTLSFV,
NTDDYVTRTSIFYHAGSSRL, FYHAGSSRLLTVGNPYFRVP, PQRHTMLCMCCKCEARIKLV,
GMHGPKATL, HGPKATLQDI, MHGPKATL or FQQLFLNTL

and/or a functionally active variant thereof, and to its use in diagnosis and therapy.

The papillomavlruses, also termed wart viruses, are double-stranded DNA viruses which possess a genome size of about 8000 base pairs and an icosahedral capsid having a diameter of approx. 55 nm. To date, more than 100 different papillomavirus types (HPV) which are pathogenic to humans are known, some of which, e.g. HPV-16, HPV-18, HPV-31, HPV-33, HPV-39, HPV-45, HPV-52 and HPV-58, are able to give rise to malignant tumors and others, e.g. HPV-6, HPV-11 and HPV-42, to benign tumors.

The papillomavirus genome can be subdivided into three regions: the first region concerns a noncoding region which contains elements for regulating transcription and replication of the virus. The second region, what is termed the E (early) region, contains different protein-encoding segments E1-E7, the E6 and E7 proteins of which, for example, are responsible for transforming epithelial cells while the E1 protein controls DNA copy number. The E6 and E7 regions are what are termed oncogenes, which are also expressed in malignantly degenerate cells. The third region, also termed the L (late) region, contains two protein-encoding segments L1 and L2 which encode structural components of the virus capsid. More than 90% of the viral capsid consists of the L1 protein, with the L1:L2 ratio in general being 30:1. Within the meaning of the present invention, the term L1 protein is understood as denoting the main papillomavirus capsid protein (Baker T. et al. (1991) Biophys. J. 60, 1445).

In more than 50% of cases, HPV-16 is associated with uterine cervical cancer (cervical carcinoma). HPV-16 is the main risk factor for the formation of cervical neoplasias. The immune system plays an important role in the progress of the disease. Thus, cellular immune responses and, in particular, antigen-specific T lymphocytes are presumably important for the defence mechanism. It has furthermore been found that the E7 gene is constitutively expressed in all layers of the infected epithelium in extremely malignant cervical intraepithelial neoplasias (CIN II/III) and cervical tumors. It is for this reason that the E7 protein in particular is regarded as being a potential tumor antigen and a target molecule for activated T cells (see, e.g., WO 93/20844). However, the cellular immune response which E7 induces in the patient does not appear to be sufficiently strong to influence the course of the disease. The immune response may possibly be augmented by means of suitable vaccines. It has been shown that expression of the L1 gene or coexpression of the L1 gene and the L2 gene can lead to the formation of capsomeres, stable capsomeres, capsids or virus-like particles (VLPS) (see, e.g., WO 93/02184, WO 94/20137 or WO 94/05792). Capsomeres are understood as meaning an oligomeric configuration which is composed of five L1 proteins. The capsomere is the basic building block from which viral capsids are constructed. Stable capsomeres are understood as meaning capsomeres which are unable to assemble themselves into capsids. Capsids are understood as meaning the papillomavirus coat, which is composed, for example, of 72 capsomeres (Baker T. et al. (1991) Biophys. J. 60, 1445). VLP is understood as meaning a capsid which resembles an intact virus morphologically and in its antigenicity. VLPs have been used to induce a humoral immune response, which is characterized by the formation of neutralizing antibodies, in various animal systems. However, the formation of virus-neutralizing antibodies directed against L1 protein and/or L2 protein is of little clinical importance when the viral infection has already taken place since it is not antibodies, but rather a virus-specific cytotoxic T cell (CTL) response which appears to be necessary for eliminating virus-infected cells. Furthermore, although VLPs are able to induce a cytotoxic T cell response, an immune response which is directed exclusively against the capsid proteins L1 and/or L2 does not appear to be suitable for combating a papillomavirus-associated tumor.

What are termed chimeric papillomavirus-like particles (CVLPs) have therefore been developed, with these particles consisting of an HPV-16 fusion protein formed between the capsid protein L1 and the potential tumor antigen E7 (WO 96/11272 and Müller, M. et al. (1997) Virology, 234, 93). The CVLPs only induced a slight humoral immune response directed against the E7 protein (Müller, M. et al. (1997), see above). However, some of the CVLPs which were tested did in fact induce the desired E7-specific cytotoxic T cell response in mice (see also Peng S. et al. (1998) Virology 240, 147-57). CVLPs are therefore of interest both for developing a vaccine and for treating already existing infections and tumors resulting therefrom since the E7 peptides which were presented by tumor cells by way of class I MHC molecules would constitute target molecules for cytotoxic T cells.

A vaccine composed of CVLPs is based on the principle of pseudoinfecting the cells with the CVLPS. This means that the CVLPs, like viruses, gain access to the cell, where they are processed into peptides, and the peptides are then loaded onto MHC class I and class II molecules and finally presented to CD8-positive and CD4-positive T cells, respectively. As a consequence of this stimulation, CD8 cells can differentiate into cytotoxic T cells and then bring about a cellular immune response; CD4 cells on the other hand develop into T helper cells and stimulate B cells to give a humoral immune response or CD8-positive T cells to give a cytotoxic immune response and can themselves induce lysis of infected cells.

Small peptides can already bind to MHC class I molecules on the cell surface and then, without any further processing, stimulate CD8-positive or CD4-positive cells to give a cellular immune response. However, a particular peptide can only be bound by particular MHC molecules. As a result of the extensive polymorphism of the MHC molecules in natural populations, a particular peptide can therefore only be bound and presented by a small proportion of a population. Within the meaning of the present invention, presentation is understood as denoting when a peptide or protein fragment binds to an MHC molecule, with this binding being able to take place, for example, in the endoplasmic reticulum, in the extracellular space, the endosomes, proendosomes, lysosomes or protolysosomes, and when this MHC molecule/peptide complex is then bound on the extracellular side of the cell membrane so that it can be specifically recognized by immune cells.

Since CVLPs induce both a cellular immune response and a humoral immune response and are not MHC-restricted, these particles are suitable, in a general manner, for developing vaccines, with an L1 moiety providing the ability to form particles and an additional antigen moiety being fused to this L1 moiety.

When such CVLPs are being developed, it is absolutely necessary to have available a functional test system which can be used to directly investigate CVLP immunogenicity. Such a test system should possess the property that CVLPs containing different antigen moieties can be investigated using the same test system. Since the cellular immune response is of crucial importance for immunological methods for treating tumors or viral diseases, the object arose of making the cellular immune response induced by type 16 or type 18 CVLPs measurable.

This object was achieved by identifying HPV-16 or HPV-18 T cell epitopes which, in combination with MHC molecules, induce a cytotoxic T cell response, for example, in vivo and in vitro. The peptides according to the invention therefore have the sequence

YLPPVPVSKVVSTDEYVART, STDEYVARTNIYYHAGTSRL, VGHPYFPIKKPNNNKILVPK,
GLQYRVFRIHLPDPNKFGFP, WACVGVEVGRGQPLGVGISG, QPLGVGISGHPLLNKLDDTE,
QLCLIGCKPPIGEHWGKGSP, LELINTVIQDGDMVDTGFGA, DMVDTGFGAMDFTTLQANKS,
VTVVDTTRSTNNSLCAAIST, TTYKNTNFKEYLRHGEEYDL, IFQLCKITLTADVMTYIHSM,
PPPGGTLEDTYRFVTSQAIA, RFVTSQAIACQKHTPPAPKE, LKKYTFWEVNLKEKFSADLD,
PLGRKFLLQAGMHGDTPTLH, YCYEQLNDSSEEEDEIDGPA, VGNPYFRVPAGGGNKQDIPK,
GGNKQDIPKVSAYQYRVFRV, SIYNPETQRLVWACAGVEIG, IYNPETQRL,
PDYLQMSADPYGDSMFFCLR, GDSMFFCLRREQLFARHFWN,
NNGVCWHNQLFVTVVDTTRS, PPPPTTSLVDTYRFVQSVAI, YRFVQSVAITCQKDAAPAEN,
PYDKLKFWNVDLKEKFSLDL, YPLGRKFLVQAGMHGPKATL, MHGPKATLQDIVLHLEPQNE,
VDLLCHEQLSDSEEENDEID, SEEENDEIDGVNHQHLPARR, SSADDLRAFQQLFLNTLSFV,
NTDDYVTRTSIFYHAGSSRL, FYHAGSSRLLTVGNPYFRVP, PQRHTMLCMCCKCEARIKLV,
GMHGPKATL, HGPKATLQDI, MHGPKATL or FQQLFLNTL.

Potential epitopes have already been published in Kast et al., (1994) Journal of Immunology 152, 3904-3912. However, this publication only shows that these peptides are able to bind to HLA A1 molecules but not that a cytotoxic T cell response can in fact be induced. Furthermore, the publication does not cite any data which demonstrate that T cells can recognize the peptides as protein constituents. It has been shown many times that peptides which bind per se to HLA molecules are not necessarily also recognized by T cells. It is furthermore known that T cells which recognize a peptide, as can be measured by the fact that the peptide can induce them to give a T cell response, nevertheless do not necessarily also recognize cells which have been loaded with entire proteins which contain the corresponding peptide. This can be explained by the fact that peptides frequently contain protease cleavage sites within which the peptides are cleaved, and therefore destroyed, while the entire proteins are being processed in the cell; as a consequence, the peptides can no longer be recognized by T cells. This problem is confirmed, for example, in Feltkamp et al. (1993), Eur. J. Immunol. 23: 2242-2249.

The present invention therefore relates to a T cell epitope having an amino acid sequence

YLPPVPVSKVVSTDEYVART, STDEYVARTNIYYHAGTSRL, VGHPYFPIKKPNNNKILVPK,
GLQYRVFRIHLPDPNKFGFP, WACVGVEVGRGQPLGVGISG, QPLGVGISGHPLLNKLDDTE,
QLCLIGCKPPIGEHWGKGSP, LELINTVIQDGDMVDTGFGA, DMVDTGFGAMDFTTLQANKS,
VTVVDTTRSTNMSLCAAIST, TTYKNTNFKEYLRHGEEYDL, IFQLCKITLTADVMTYIHSM,
PPPGGTLEDTYRFVTSQAIA, RFVTSQAIACQKHTPPAPKE, LKKYTFWEVNLKEKFSADLD,
PLGRKFLLQAGMHGDTPTLH, YCYEQLNDSSEEEDEIDGPA, VGNPYFRVPAGGGNKQDIPK,
GGNKQDIPKVSAYQYRVFRV, SIYNPETQRLVWACAGVEIG, IYNPETQRL,
PDYLQMSADPYGDSMFFCLR, GDSMFFCLRREQLFARHFWN, NNGVCWHNQLFVTVVDTTRS,
PPPPTTSLVDTYRFVQSVAI, YRFVQSVAITCQKDAAPAEN, PYDKLKFWNVDLKEKFSLDL,
YPLGRKFLVQAGMHGPKATL, MHGPKATLQDIVLHLEPQNE, VDLLCHEQLSDSEEENDEID,
SEEENDEIDGVNHQHLPARR, SSADDLRAFQQLFLNTLSFV, NTDDYVTRTSIFYHAGSSRL,
FYHAGSSRLLTVGNPYFRVP, PQRHTMLCMCCKCEARIKLV, GMHGPKATL, HGPKATLQDI,
MHGPKATL or FQQLFLNTL

and/or a functionally active variant thereof.

A functionally active variant of the T cell epitopes according to the invention is understood as being a T cell epitope which, in a T cell cytotoxicity test system (see, for example, examples 2-5 of the present invention), possesses a cytotoxicity, as measured against the cytotoxicity of the T cell epitope according to the invention, which corresponds to at least the sum of the mean of the negative controls and three times the standard deviation, preferably of at least approx. 30%, in particular at least approx. 50% and particularly preferably of at least approx. 80%.

An example of a preferred variant is a T cell epitope having a sequence homology with the T cell epitopes according to the invention of at least approx. 65%, preferably at least approx. 75% and, in particular, at least approx. 85% on the amino acid level. Other preferred variants are also T cell epitopes which possess structural homology with the T cell epitopes according to the invention. Such epitopes can be found by generating specific T cells directed against the T cell epitopes according to the invention (DeBruijn M. L. et al. (1991) Eur. J. Immunol. 21, 2963-70; and DeBruijn M. L. (1992) Eur. J. Immunol. 22, 3013-20) and, for example, synthetically prepared peptides being tested, following selection, for recognition by the peptide-specific T cells (see examples). In particular, T cell epitopes are understood as meaning cytotoxic T cell epitopes or T helper cell epitopes (T_{H}, T_{H1} or T_{H2}) However, noncytotoxic T cells, which are likewise able to recognize MHC I molecules, are also known such that noncytotoxic T cell epitopes are also included as variants of the present invention.

Another embodiment of the present invention is a T cell epitope which is part of a compound, with the compound not being any naturally occurring L1 protein derived from a papillomavirus and, in the case of an HPV-16 T cell epitope, not being an exclusively N-terminal or exclusively C-terminal deletion mutant of a naturally occurring L1 protein derived from a papillomavirus. For example, the compound can be a fusion of identical or different T cell epitopes according to the invention.

In one particular embodiment, a T cell epitope according to the invention, and/or a functionally active variant, can be present in an L1 protein derived from a different papillomavirus or in a chimeric L1 protein, for example an HPV18 L1E7 fusion protein or HPV16 L1E7 fusion protein. Such a compound according to the invention may possess the ability to form CVLPs.

Said T cell epitope can preferably, as part of a compound, be a polypeptide which, in a preferred manner, contains further amino acid sequences, in particular a fusion protein. In particular, the compound can be a polypeptide of at least approx. 50 amino acids, preferably of at least approx. 35 amino acids, in particular of at least approx. 20 amino acids and, in a particularly preferred manner, of at least approx. 9-12 amino acids in length.

In order to detect the compound or modify its activity in binding to T cells, it can contain a chemical, radioactive isotope, nonradioactive isotope and/or fluorescent labeling of the T cell epitope and/or of the said fusion protein.

Examples of chemical substances which are known to the skilled person and which are suitable for a chemical label according to the invention are: biotin, FITC (fluorescein isothiocyanate) and streptavidin.

One possible embodiment is that a peptide is modified such that it contains at least one lysine. Biotin or FITC (fluorescein isothiocyanate) is then coupled to this lysine in the manner known to the skilled person. A peptide which has been modified in this way is bound to an appropriate MHC molecule or to a cell containing appropriate MHC molecules. The peptide can then be detected by way of labeled avidin or streptavidin or directly using the fluorescence of the FITC.

Examples of isotopes which are known to the skilled person and which are suitable for a radioactive isotope label according to the invention are: ³H, ¹²⁵I, ³²P, ³³P and ¹⁴C.

Examples of isotopes which are known to the skilled person and which are suitable for a nonradioactive isotope label according to the invention are: ²H and ¹³C.

Examples of fluorescent substances which are known to the skilled person and which are suitable for a fluorescent label according to the invention are: ¹⁵²Eu, fluorescein isothiocyanate, rhodamine, phycoerythrin, phycocyanin, allophycocyanin, o-phthaldehyde and fluorescamine.

The skilled person is familiar with other labels which are not cited here but which can also be used for labeling within the meaning of this invention.

Examples of chemical modifications according to the invention which are known to the skilled person are the transfer of acetyl, phosphate and/or monosaccharide groups.

Polypeptides according to the invention having an amino acid length of approx. 50 can be prepared, for example, by means of chemical peptide synthesis. Longer polypeptides are preferably produced recombinantly.

The present invention therefore also relates to a nucleic acid, for example for expressing said T cell epitope or the compounds, which, for example, contains the following components: (a) at least one regulatory element and (b) at least one nucleic acid which encodes an amino acid sequence of the compound according to the invention. Said nucleic acid construct is preferably composed of DNA or RNA. Suitable regulatory elements make possible, for example, constitutive, regulable, tissue-specific, cell cycle-specific or metabolically specific expression in eukaryotic cells or constitutive, metabolically specific or regulable expression in prokaryotic cells. According to the present invention, regulable elements are promoters, activator sequences, enhancers, silencers and/or repressor sequences.

Examples of suitable regulable elements which make possible constitutive expression in eukaryotes are promoters which are recognized by RNA polymerase III or viral promoters, such as the CMV enhancer, the CMV promotor, the SV40 promotor and viral promotor and activator sequences which are derived, for example, from HBV, HCV, HSV, HPV, EVB HTLV and HIV.

Examples of regulable elements which make possible regulable expression in eukaryotes are the tetracycline operator in combination with an appropriate repressor (Gossen M. et al (1994) Curr. Opin. Biotechnol. 5, 516-20).

Examples of regulable elements which make possible tissue-specific expression in eukaryotes are promotors of activator sequences composed of promotors or enhancers derived from those genes which encode proteins which are only expressed in certain cell types.

Examples of regulable elements which make possible cell cycle-specific expression in eukaryotes are the promotors of the following genes: cdc25C, cyclin A, cyclin E, cdc2, E2F, B-myb and DHFR (Zwicker J. and Müller R. (1997) Trends Genet. 13, 3-6).

Examples of regulable elements which make possible metabolically specific expression in eukaryotes are promotors which are regulated by hypoxia, by glucose lack, by phosphate concentration or by heat shock.

The nucleic acid according to the invention can, for example, also be used for producing a DNA vaccine. For this, the sequences encoding these epitopes and other already known papillomavirus epitopes can be joined together, in any arbitrary sequence, directly or with several nucleotides in between, in an open reading frame. In addition, non-papillomavirus DNA sequences, which are used, for example, for targeting the resulting polypeptide chain intracellularly, in particular into the endoplasmic reticulum, into the endosomes or into the lysosomes, can be added on.

In order to enable said nucleic acid to be introduced into a eukaryotic or prokaryotic cell by transfection, transformation or infection, the nucleic acid can be present as a plasmid, or as a part of a viral or nonviral vector.

The present invention therefore also relates to a vector, in particular an expression vector, which contains a nucleic acid according to the invention. Particularly suitable viral vectors in this connection are: baculoviruses, vaccinia viruses, adenoviruses, adenoassociated viruses and herpesviruses. Particularly suitable nonviral vectors in this connection are: virosomes, liposomes, cationic lipids and polylysine-conjugated DNA.

The present invention furthermore additionally relates to a cell which contains, and is preferably presenting, at least one T cell epitope. In one particular embodiment, the cell is transfected, transformed or infected with one of the vectors according to the invention. For example, this cell expresses the polypeptide according to the invention under conditions which are known to the skilled person and which lead to the activation of the regulable elements which are being used at the time. The polypeptide can then be isolated from this cell and be purified, for example using one of the abovementioned labels. Prokaryotic and eukaryotic cells, in particular bacterial cells, such as E. coli, yeast cells, such as S. cerevisiae, insect cells, such as Spodoptera frugiperda cells (Sf-9) or Trichoplusia ni cells, or mammalian cells, such as COS cells or HeLa cells, are suitable for carrying out the recombinant preparation. The compounds according to the invention which have been expressed can then be purified using standard methods.

A preferred embodiment is to use the cell which is expressing the polypeptide according to the invention itself, with, in a particularly preferred embodiment, the cell presenting parts of the polypeptide according to the invention, by way of MHC-l molecules, on the cell surface. Antigen-presenting cells, such as B cells, macrophages, dendritic cells, fibroblasts or other HLA A2.01-positive cells, in a preferred embodiment JY, T2 or CaSki cells or EBV-transformed B cell lines (BLCLs), are suitable for producing a cell according to the invention. The cells according to the invention, which present a polypeptide containing a T cell epitope, can be used as target cells for restimulating immune cells, in particular T cells, and/or for measuring the activation of T cells. Within the meaning of the present invention, a target cell is to be understood as being a cell which presents a T cell epitope by way of MHC molecules and thereby specifically elicits T cell activation, in particular a cytotoxic T cell reaction directed against the cell.

Furthermore, the compound containing a T cell epitope can be part of a complex which is characterized by the fact that the compound is linked covalently, or by way of hydrophobic interactions, ionic bonds or hydrogen bonds to at least one further species such as peptides, proteins, peptoids, linear or branched oligosaccharides or polysaccharides and nucleic acids.

The present invention therefore relates to a complex which contains a T cell epitope or a compound and at least one further compound. A preferred embodiment is that the polypeptide is present in combination with MHC class I molecules, for example as HLA A2.01 (or else HLA A1, HLA A24, etc.) tetramer. Human MHC class I molecules are particularly preferred. For example, the technique of Altman J. D. et al. (1996, Science 274, 94-6) can be used, for example, to prepare HLA A2.01 tetramers containing the appropriate bound peptides which are able to bind to the T cell receptors of peptide-specific cytotoxic T cells.

Another embodiment is that of immobilizing the compound according to the invention, or said complex, on support materials. Examples of suitable support materials are ceramic, metal, in particular precious metal, glass, plastics, crystalline materials or thin layers of the support, in particular of said materials, or (bio)molecular filaments such as cellulose or structural proteins.

For purifying the complex according to the invention, one component of the complex can additionally contain a protein tag. Protein tags according to the invention make possible, for example, high-affinity absorption to a matrix, stringent washing with suitable buffers without the complex being eluted to any significant degree and subsequent selective elution of the absorbed complex. Examples of protein tags which are known to the skilled person are a (HIS)₆ tag, a Myc tag, a FLAG tag, a hemaglutenin tag, a glutathione transferase (GST) tag, intein containing an affinity chitin-binding tag or maltose-binding protein (MBP) tag. The protein tags according to the invention can be located N-terminally, C-terminally and/or internally.

The present invention also relates to a method for detecting in vitro the activation of T cells using at least one compound containing a T cell epitope. Such a method preferably consists of three steps:

a) In a first step, cells are stimulated with at least one T cell epitope according to the invention, preferably with at least one compound containing a T cell epitope according to the invention. This compound can denote at least one compound according to the invention containing a T cell epitope, at least one complex according to the invention containing a T cell epitope, at least one capsomere, at least one stable capsomere, at least one VLP, at least one CVLP and/or at least one virus. In a preferred embodiment, immune cells are stimulated by being incubated with CVLPs. This stimulation can take place, for example, in the form of a vaccination or by incubating immune cells with CVLPs in vitro. Immune cells which have been stimulated in this way are isolated, for example after a vaccination or in the case of a tumor patient, from the blood, from tumors or from lymph nodes, and/or cultured.

b) In a second step, cells are incubated with at least one T cell epitope according to the invention, at least one compound according to the invention containing a T cell epitope, at least one target cell which is presenting a T cell epitope and/or with at least one complex according to the invention.

c) In a third step, the activation of T cells is determined. Methods which are suitable for this purpose are, for example, those of detecting the production or secretion of cytokines by the T cells, detecting the expression of surface molecules on T cells, detecting the lysis of target cells or detecting the proliferation of cells. Examples of procedures which are suitable for this purpose are a cytokine assay (chapter 6.2 to 6.24 in Current Protocols in Immunology (1999), edited by Coligan J. E., Kruisbeek A. M., Margulies D. H., Shevach E. M. and Strober W., John Wiley & Sons), ELISPOT (chapter 6.19 in Current Protocols in Immunology, see above), a ⁵¹Cr release test (chapter 3.11 in Current Protocols in Immunology, see above) or detection of proliferation (chapter 3.12 in Current Protocols in Immunology, see above). Depending on the procedure used in this connection, it is also possible to differentiate between the immune cells such as cytotoxic T cells, T helper cells, B cells, NK cells and other cells. The use of compounds according to the invention, complexes and/or cells which contain labels according to the invention makes it possible to detect T cells which recognize the T cell epitope by means of detecting the binding of labeled compounds, complexes and/or cells to the T cells. In a preferred embodiment, the binding of MHC/polypeptide complexes according to the invention to the surface of the T cells is detected. This can be effected by the MHC complexes themselves being labeled, for example being fluorescence-labeled, or by an MHC-specific, labeled, for example fluorescence-labeled, antibody being used in a further step in order, once again, to detect the MHC complexes. The fluorescence labeling of the T cells can then be measured and analyzed, for example, in a fluorescence-activated cell sorter (FACS). Another possibility for detecting the binding of the complexes to the T cells is once again that of measuring the activation of T cells (cytokine assay, Elispot, ⁵¹Cr release test or proliferation, see above). However, simultaneous stimulation of coreceptors (e.g. CD28), for example by means of coreceptor-specific antibodies (anti-CD28) and/or other nonspecific activators (IL-2) is required to do this.

The present invention also relates to a method which contains an additional step a′), which is inserted after step a).

a′) In this additional step a′), which follows step a), the isolated or cultured cells are cocultured with at least one target cell loaded with at least one T cell epitope according to the invention, with at least one compound according to the invention containing a T cell epitope, at least one complex according to the invention containing a T cell epitope, at least one capsomere, at least one stable capsomere, at least one VLP, at least one CVLP and/or at least one virus, with at least one complex according to the invention containing a T cell epitope, and/or at least one target cell which is presenting a T cell epitope, for at least approx. 8 weeks, in particular for at least approx. 1 week, before step b) follows.

Coculturing is to be understood as meaning the growth of the cells:

(i) in the presence of at least one target cell loaded with at least one T cell epitope according to the invention, with a compound according to the invention containing a T cell epitope, at least one complex according to the invention containing a T cell epitope, at least one capsomere, at least one stable capsomere, at least one VLP, at least one CVLP, and/or at least one virus,

(ii) in the presence of at least one complex according to the invention containing a T cell epitope,

(iii) in the presence of at least one target cell which is presenting a T cell epitope according to the invention,

in the same growth medium and the same tissue culture receptacle.

The present invention also relates to a method for preparing a T cell epitope-presenting target cell. In this connection, it is possible to load the target cell with combinations of different T cell epitopes. In a preferred embodiment, the target cell is incubated with at least one T cell epitope according to the invention, with at least one compound containing a T cell epitope and/or at least one complex containing a T cell epitope. In a particularly preferred embodiment, the target cell is incubated in growth medium which contains polypeptides according to the invention or with MHC class I complexes containing bound polypeptides according to the invention. The MHC class I complexes can, for example, be present as HLA A2.01 tetramers. As a rule, a tetramer binds four peptides in this connection. These peptides can either be identical or represent different species of peptides. In another preferred embodiment, the target cell is transfected, transformed and/or infected with a nucleic acid according to the invention and/or a vector according to the invention. In a particularly preferred embodiment, the target cell is infected with a vaccinia virus vector. The method according to the invention is carried out using antigen-presenting cells, for example using B cells, macrophages, dendritic cells, embryonic cells or fibroblasts or other HLA-positive cells, in one embodiment using JY, T2 or CaSki cells or EBV-transformed B cell lines.

The CVLPs which are used contain a papillomavirus L1 protein or variants thereof, in particular HPV16 L1 protein, and, though not necessarily, a protein, or variants thereof, which is/are heterologous to L1. The two proteins can be present in the form where they are bonded directly or indirectly. Within the meaning of the invention, bonded directly means that a covalent bond exists between the two proteins, for example a peptide bond or a disulfide bond. Indirectly bonded means that the proteins are bonded by way of noncovalent bonds, for example hydrophobic interactions, ionic bonds or hydrogen bonds. In another embodiment, the CVLPs contain a papillomavirus L2 protein in addition to L1 protein or variants thereof.

An example of a preferred embodiment of the L1 protein of the present invention is represented by L1 proteins which contain one or more deletions, in particular a C-terminal deletion. A C-terminal deletion has the advantage that the efficiency of forming virus-like particles can be increased since the nuclear localization signal, which is located at the C-terminus, is deleted. The C-terminal deletion therefore preferably amounts to up to approx. 35 amino acids, in particular from approx. 25 to approx. 35 amino acids, especially from approx. 32 to approx. 34 amino acids. For example, a C-terminal deletion of the HPV16 L1 protein of 32 amino acids in length is sufficient to be able to increase the formation of virus-like particles by at least approx. tenfold. Furthermore, the L1 protein can carry one or more mutations or the L1 moiety can be composed of L1 proteins from different papillomaviruses. The characteristic possessed in common by the L1 proteins according to the invention is that they make possible the formation of VLPs or CVLPs and that they contain at least one T cell epitope according to the invention.

In a preferred embodiment, the L1 protein, or variants thereof, and the protein which is heterologous to L1 form a fusion protein. This also includes heterologous proteins which are composed of several different proteins or parts thereof. These can also, for example, be epitopes, in particular cytotoxic T cell epitopes of proteins. In this connection, epitopes within the meaning of the invention can also be a part of a synthetic polypeptide having a length of approx. 50 amino acids, preferably of at least approx. 35 amino acids, in particular of at least approx. 20 amino acids and, in a particularly preferred manner, of at least approx. 9 amino acids.

Preference is given to L1-heterologous proteins which are derived from a viral protein, for example derived from HIV, HBV or HCV, preferably from papillomaviruses, in particular from human papillomaviruses.

In a preferred embodiment, this L1-heterologous protein is a papillomavirus E protein, preferably an E6 protein and/or E7 protein. Particular preference is given to the E protein being a deleted E protein, preferably a C-terminally deleted E protein, in particular a C-terminally deleted E7 protein, since, in combination with deleted L1 protein, these constructs are preferentially able to form virus-like particles. Particular preference is given to deletions of up to 55 amino acids, preferably of from approx. 5 to approx. 55 amino acids, particularly of from approx. 38 to approx. 55 amino acids.

In another embodiment, the L1-heterologous protein can be derived from antigens derived from nonviral pathogens. They can also be derived from autoimmune antigens, such as, for example, thyroglobulin, myelin basic protein or zona pellucida glycoprotein 3 (ZP₃), which are associated with specific autoimmune diseases, such as, for example, thyroiditis, multiple sclerosis, oophoritis or rheumatoid arthritis. In a preferred embodiment, the L1-heterologous protein is derived from tumor antigens, preferably melanoma antigens, such as MART, ovarian carcinoma antigens, such as Her2 neu (c-erbB2), BCRA-1 or CA125, colori carcinoma antigens, such as CA125, or mammary carcinoma antigens, such as Her2 neu (c-erbB2), BCRA-1 or BCRA-2.

This invention also relates to a method for the invitro detection of the activation of T cells which are obtained by preparing them from samples. This method makes it possible to determine whether papillomavirus L1 protein-specific cytotoxic T cells are present in a sample, for example a blood sample from a patient, or in tumors or lymph nodes in a tumor patient. This detection method contains the following steps:

a″) In a first step, cells are obtained, for example by withdrawing blood from a patient or by dissecting tumors or lymph nodes, for example. The cells are then taken up in growth medium and cultured.

b) In a second step, cells are incubated with at least one target cell which is presenting a T cell epitope or with at least one complex which includes, as a component, a compound containing a T cell epitope.

c) In a third step, the activation of T cells is determined. Methods which are suitable for this purpose are, for example, those of detecting the production or secretion of cytokines by the T cells, detecting the expression of surface molecules on T cells, detecting the lysis of target cells or detecting the proliferation of cells. Examples of procedures which are suitable for this purpose are a cytokine assay (chapter 6.2 to 6.24 in Current Protocols in Immunology (1999), edited by Coligan J. E., Kruisbeek A. M., Margulies D. H., Shevach E. M. and Strober W., John Wiley & Sons), ELISPOT (chapter 6.19 in Current Protocols in Immunology, see above), a ⁵¹Cr release test (chapter 3.11 in Current Protocols in Immunology, see above) or detection of proliferation (chapter 3.12 in Current Protocols in Immunology, see above). Depending on the procedure employed, it is also possible, in this connection, to differentiate between the immune cells such as cytotoxic T cells, T helper cells, B cells, NK cells and other cells. The use of compounds according to the invention, complexes and/or cells which contain labels makes it possible to detect T cells which recognize the T cell epitope by means of detecting the binding of labeled compounds, complexes and/or cells to the T cells. In a preferred embodiment, the binding of MHC/polypeptide complexes according to the invention to the surface of the T cells is detected. This can be carried out by the MHC complexes themselves being labeled, for example fluorescence-labeled, or by an MHC-specific, labeled, for example fluorescence-labeled, antibody being used in a further step in order, once again, to detect the MHC complexes. The fluorescence labeling of the T cells can then be measured and analyzed, for example in a fluorescence-activated cell sorter (FACS). Another possibility for detecting the binding of the complexes to the T cells is once again that of measuring the activation of T cells (cytokine assay, Elispot, ⁵¹Cr release test or proliferation, see above). However, the simultaneous stimulation of coreceptors (e.g. CD28), for example by means of coreceptor-specific antibodies (anti-CD28) and/or other nonspecific activators (IL-2) is required to do this.

The present invention also relates to a method which contains an additional step a′) which is inserted after step a″).

a′) In this additional step a′), which follows the step a″), the isolated or cultured cells are cocultured with at least one target cell loaded with at least one T cell epitope according to the invention, with a compound according to the invention containing a T cell epitope, at least one complex according to the invention containing a T cell epitope, at least one capsomere, at least one stable capsomere, at least one VLP, at least one CVLP and/or at least one virus, with at least one complex according to the invention containing a T cell epitope, and/or at least one target cell which is presenting a T cell epitope, for at least approx. 8 weeks, in particular for at least approx. 1 week, before step b) follows.

Coculturing is to be understood as the growth of the cells:

(i) in the presence of at least one target cell loaded with at least one T cell epitope according to the invention, with a compound according to the invention containing a T cell epitope, at least one complex according to the invention containing a T cell epitope, at least one capsomere, at least one stable capsomere, at least one VLP, at least one CVLP, and/or at least one virus,

(ii) in the presence of at least one complex according to the invention containing a T cell epitope,

(iii) in the presence of at least one target cell which is presenting a T cell epitope,

in the same growth medium and the same tissue culture receptacle.

The invention also relates to a test system (kit) for the in-vitro detection of the activation of T cells comprising:

a) at least one T cell epitope according to the invention, at least one compound according to the invention, at least one vector according to the invention, at least one cell according to the invention and/or at least one complex according to the invention, and

b) effector cells of the immune system, preferably T cells, in particular cytotoxic T cells or T helper cells.

In one particular embodiment, the test system is used for determining the L1 protein-specific cytotoxic T cells which are present, for example, in a blood sample from a patient or in tumors or lymph nodes in a tumor patient. In this case, the cells described in b) are control cells which are present in the test system and whose activation by the first kit component, i.e. the substances mentioned under a), serves as standard. The activation which is observed in this reaction is compared with the T cell activation by kit component a) of cells isolated from patients.

In another preferred embodiment, the test system is used, for example, for determining the L1 protein-specific antigenicity of a compound containing a T cell epitope, of a complex containing a T cell epitope, of a capsomere, of a stable capsomere, of a VLP, of a CVLP and/or of a virus. In this case, the substances described in a) are control substances whose activating effect on the second kit component, i.e. the cells mentioned under b), serves as standard. The activation which is observed in this reaction is compared with the activating effect of a compound containing a T cell epitope, a complex containing a T cell epitope, a capsomere, a stable capsomere, a VLP, a CVLP and/or a virus on kit component b).

The invention furthermore relates to the use of at least one T cell epitope according to the invention, at least one compound according to the invention containing a T cell epitope, at least one vector according to the invention containing a nucleic acid encoding a compound containing a T cell epitope, at least one cell according to the invention containing a T cell epitope and/or at least one complex according to the invention containing a T cell epitope for inducing or for detecting an immune response.

Cells which are presenting at least one of the molecules according to the invention by way of their MHC class I molecules are particularly suitable for stimulating immune cells both in vitro and in vivo. Examples of cells which are suitable for antigen presentation are B cells, dendritic cells, macrophages, fibroblasts or other HLA-positive cells which are able to achieve stimulation of specific T cells by being cultured together with immune cells.

In one particular embodiment, a compound according to the invention, for example an HPV18 L1E7 fusion protein which additionally contains a T cell epitope according to the invention, can be used for detecting an immune response. Such a compound according to the invention may possess the ability to form CVLPs.

The invention also relates to a pharmaceutical or diagnostic agent which comprises at least one T cell epitope according to the invention, at least one compound according to the invention containing a T cell epitope, at least one vector containing a nucleic acid encoding a compound containing a T cell epitope, at least one cell according to the invention containing a T cell epitope and/or at least one complex according to the invention containing a T cell epitope, and, where appropriate, a pharmaceutically acceptable carrier.

Examples of carriers which are known to the skilled person are glass, polystyrene, polypropylene, polyethylene, dextran, nylon, amylase, natural or modified cellulose, polyacrylamides, agarose, aluminum hydroxide and magnitide.

A pharmaceutical or diagnostic agent according to the invention can be present in solution, be bound to a solid matrix and/or be mixed with an adjuvant.

The pharmaceutical or diagnostic agent can be administered in a variety of ways. Examples of administration forms which are known to the skilled person are parenteral, local and/or systemic administration by means, for example, of oral, intranasal, intravenous, intramuscular and/or topical administration. The preferred administration form is influenced, for example, by the natural route of infection of the given papillomavirus infection. The quantity which is administered depends on the age, weight and general state of health of the patient and on the type of papillomavirus infection. The pharmaceutical or diagnostic agent can be administered in the form of capsules, solution, suspension, elixir (for oral administration) or sterile solutions or suspensions (for parenteral or intranasal administration). Salt solution of phosphate-buffered salt solution can be used, for example, as an inert and immunologically acceptable carrier. The pharmaceutical is administered in therapeutically effective quantities. This means quantities which are sufficient to elicit a protective immunological response.

In one particular embodiment, a compound according to the invention, for example an HPV18 L1E7 fusion protein which additionally contains a T cell epitope according to the invention, can be used as a pharmaceutical or diagnostic agent. Such a compound according to the invention may possess the ability to form CVLPs.

The figures and the following examples are intended to clarify the invention without restricting it.

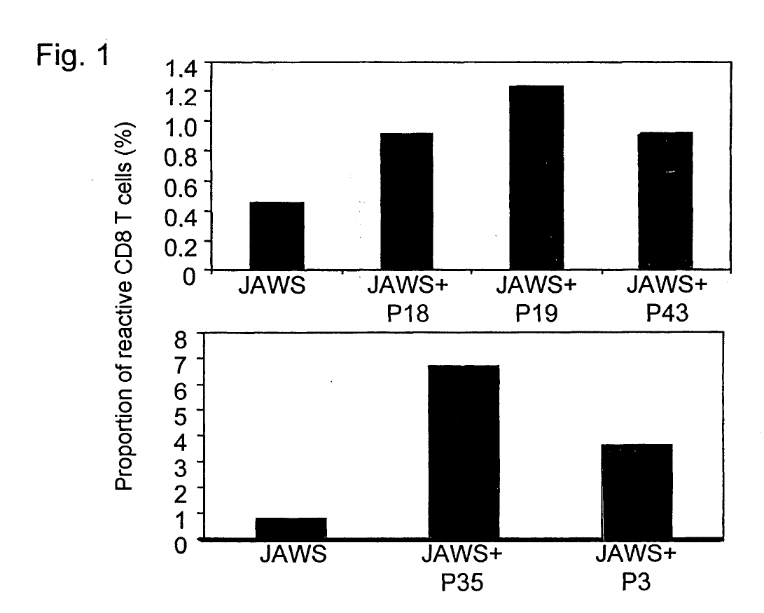

FIG. 1 shows the diagrammatic analysis of two FACScan experiments following restimulation of specific murine T cells with JAWS cells which are presenting different peptides. The name of the respective peptide is given on the X axis; JAWS cells without peptide were only incubated with buffer and served as a negative control. The proportion of CD8-positive T cells which were classified as being reactive on the basis of IFN_{γ} expression in the FACScan experiment is plotted on the Y axis.

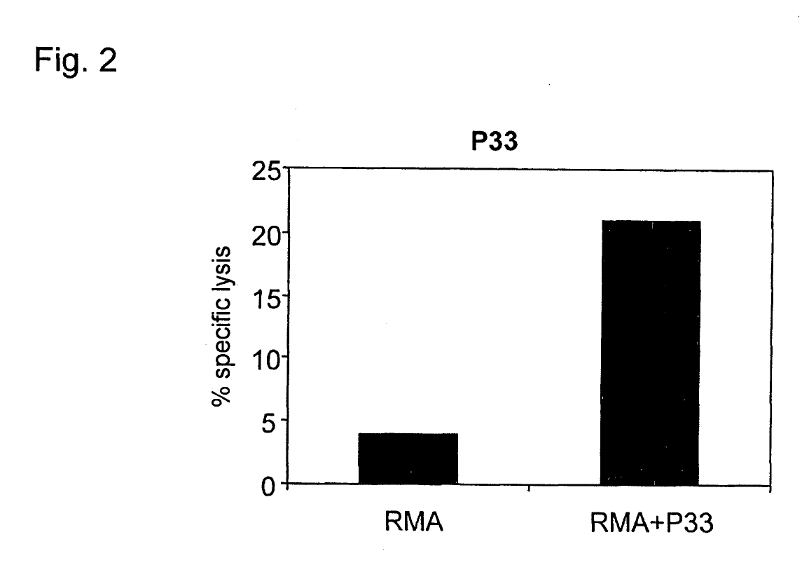

FIG. 2 shows the diagrammatic analysis of a ⁵¹Cr release experiment following the loading of RMA cells with the peptide P33 (target cells). The target cells were lysed by T cells (effector cells) which were stimulated with peptides P1 to P43, with the ratio of the effector cells employed to the target cells employed being 20. RMA cells incubated with buffer (negative control) and, respectively, RMA cells incubated with P33 peptide are plotted on the X axis. The % of the target cells which were specifically lysed, as determined by the release of ⁵¹Cr from the target cells, is plotted on the Y axis. The % values were calculated using the formula given in example 4.

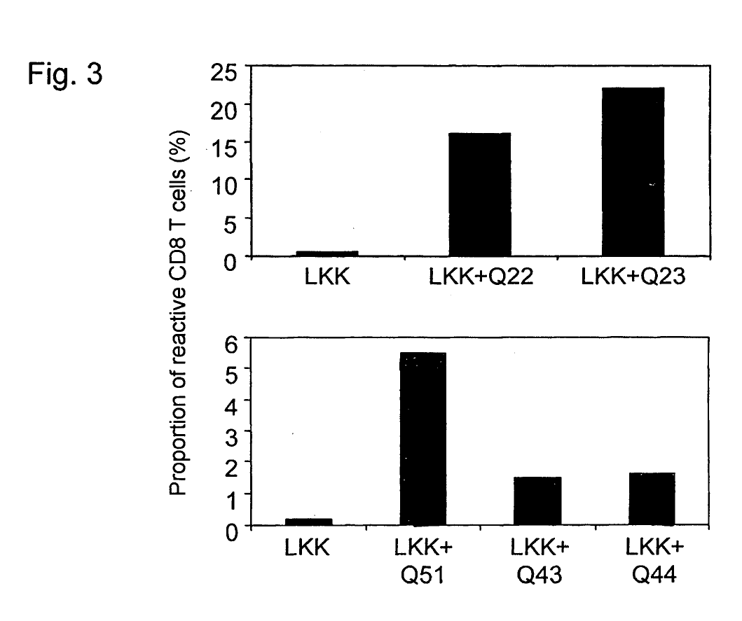

FIG. 3 shows the diagrammatic analysis of two FACScan experiments following restimulation of specific murine T cells with LKK cells which are presenting different peptides. The name of the respective peptide is given on the X axis; LKK cells without peptide were only incubated with buffer and served as a negative control. The proportion of the CD8-positive T cells which were classified in the FACScan experiment as being reactive on the basis of IFN_{γ} expression is plotted on the Y axis.

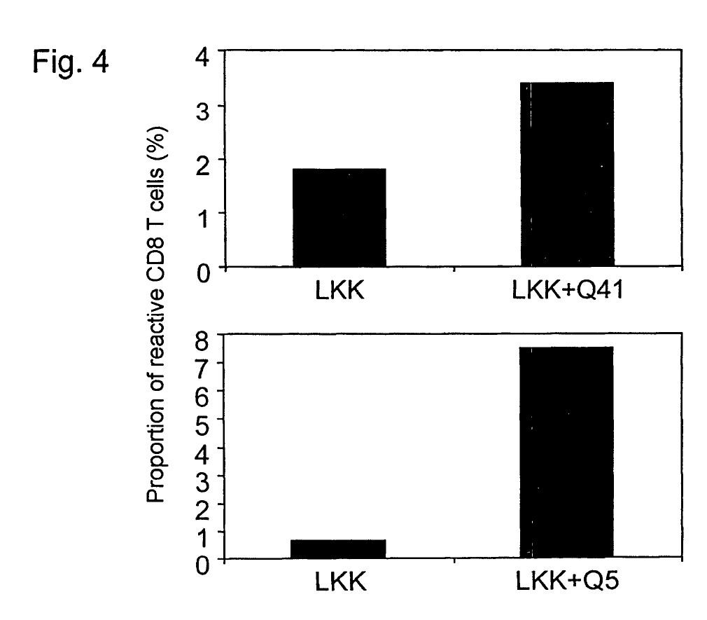

FIG. 4 shows the diagrammatic analysis of two FACScan experiments following restimulation of specific murine T cells with LKK cells which are presenting different peptides. The name of the respective peptide is given on the X axis; LKK cells without peptide were only incubated with buffer and served as a negative control. The proportion of the CD8-positive T cells which were classified in the FACScan experiment as being reactive on the basis of IFN_{γ} expression is plotted on the Y axis.

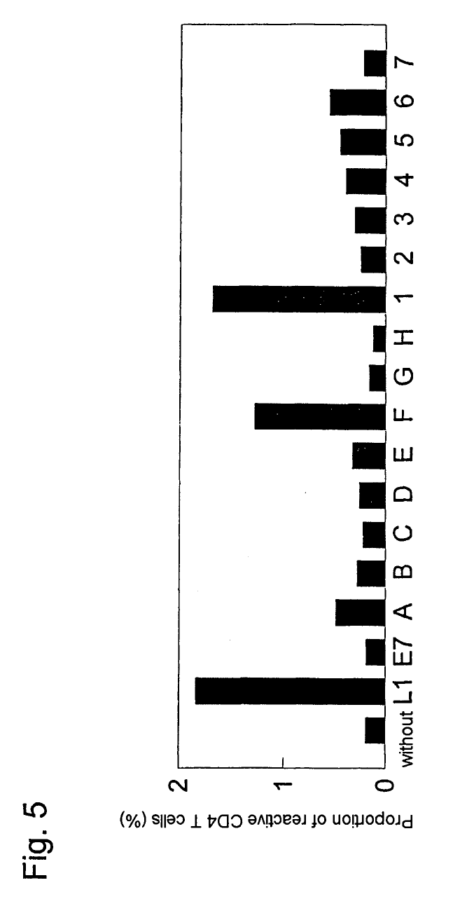

FIG. 5 shows the diagrammatic analysis of a FACScan experiment following restimulation of specific human T cells with donor-identical BLCL which are presenting different HPV18 peptide pools. The name of the respective peptide pool is given on the X axis; “without” stands for BLCL which are only incubated with buffer (negative control), while L1 and E7 stand, respectively, for BLCL which are incubated with HPV18 L1 peptide pool and HPV18 E7 peptide pool, respectively (positive controls). The proportion of CD4-positive T cells which were classified in the FACScan experiment as being reactive on the basis of IFN_{γ} expression is plotted on the Y axis.

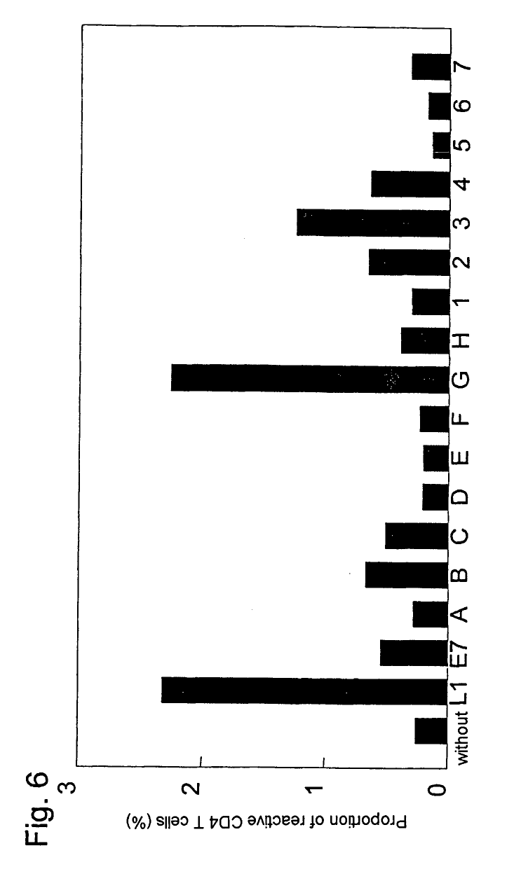

FIG. 6 shows the diagrammatic analysis of a FACScan experiment following restimulation of specific human T cells with donor-identical BLCL which are presenting different HPV18 peptide pools. The name of the respective peptide pool is given on the X axis; “without” stands for BLCL which are only incubated with buffer (negative control), while L1 and E7 stand, respectively, for BLCL which are incubated with HPV18 L1 peptide pool and HPV18 E7 peptide pool, respectively (positive controls). The proportion of CD4-positive T cells which were classified in the FACScan experiment as being reactive on the basis of IFN_{γ} expression is plotted on the Y axis.

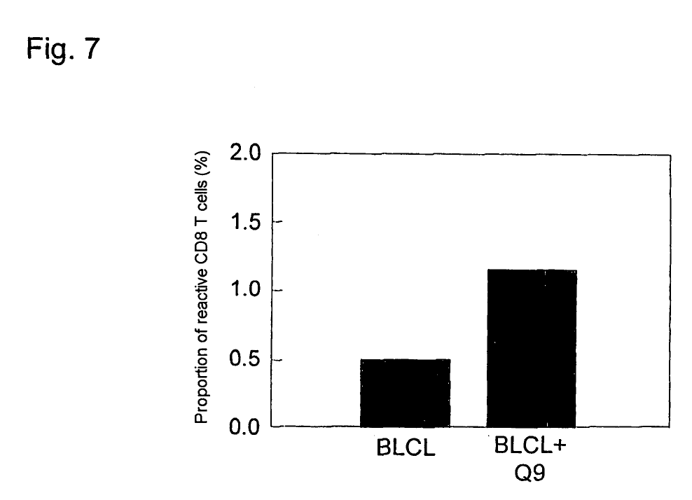

FIG. 7 shows the diagrammatic analysis of a FACScan experiment following restimulation of specific human T cells with donor-identical BLCL which are presenting the Q9 peptide. The name of the respective peptide is given on the X axis; BLCL stands for BLCL which are only incubated with buffer (negative control). The proportion of the CD8-positive T cells which were classified in the FACScan experiment as being reactive on the basis of IFN_{γ} expression is plotted on the Y axis.

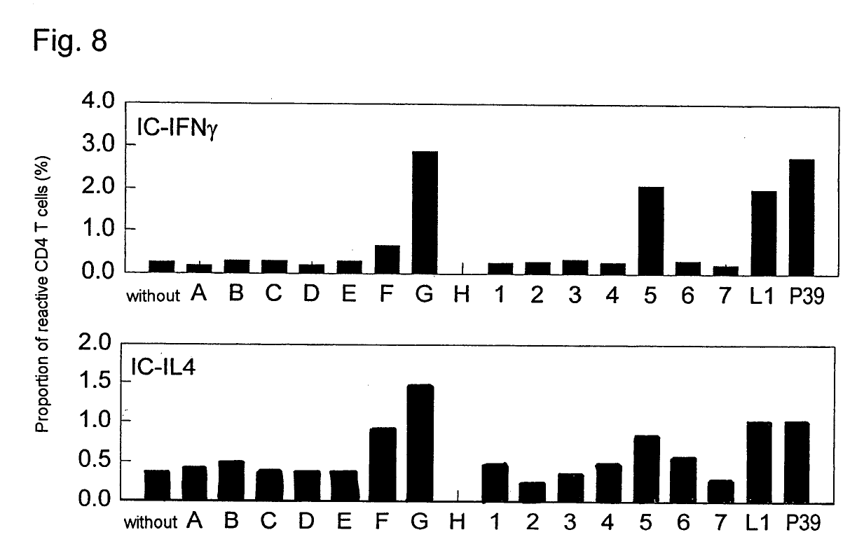

FIG. 8 shows the diagrammatic analysis of two FACScan experiments following restimulation of specific human T cells with donor-identical BLCL which are presenting different HPV16 peptide pools or the P39 peptide. The name of the respective peptide pool (A to H, 1 to 7) or of the respective peptide (P39) is given on the X axis; “without” stands for BLCL which are only incubated with buffer (negative control), while L1 stands for BLCL which are incubated with HPV16 L1 peptide pool (positive control). The proportion of the CD4-positive T cells which were classified in the FACScan experiments as being reactive, and consequently as being T helper 1 cells (T_{H1}), on the basis of IFN_{γ} expression is plotted on the Y axis in the upper part of FIG. 8. The proportion of CD4-positive T cells which were classified in the FACScan experiment as being T helper 2 cells (T_{H2}) on the basis of IL-4 expression is plotted on the Y axis in the lower part of FIG. 8.

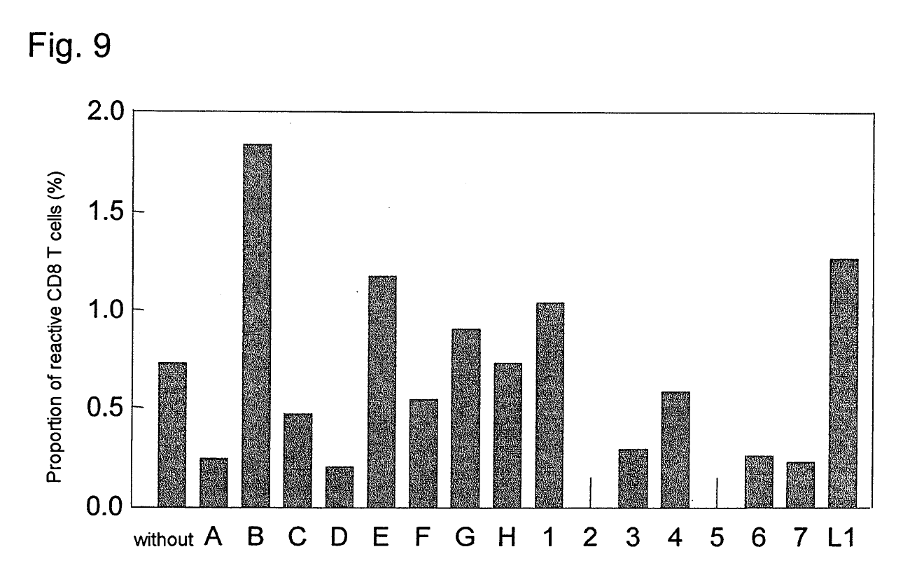

FIG. 9 shows the diagrammatic analysis of a FACScan experiment following restimulation of specific human T cells with donor-identical BLCL which are presenting different HPV16 peptide pools. The name of the respective peptide pool is given on the X axis; “without” stands for BLCL which were only incubated with buffer (negative control), while L1 stands for BLCL which were incubated with HPV16 L1 peptide pool (positive controls). The proportion of CD8-positive T cells which were classified in the FACScan experiment as being reactive on the basis of IFN_{γ} expression is plotted on the Y axis.

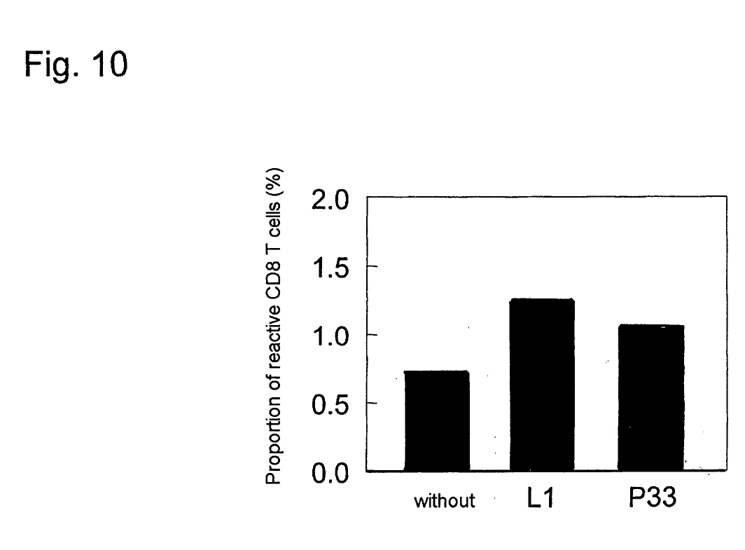

FIG. 10 shows the diagrammatic analysis of a FACScan experiment following restimulation of specific human T cells with donor-identical BLCL which are presenting P33 peptide. The name of the peptide is given on the X axis; “without” stands for BLCL which were only incubated with buffer (negative control), while L1 stands for BLCL which were incubated with HPV16 L1 peptide pool (positive controls). The proportion of CD8-positive T cells which were classified in the FACScan experiment as being reactive on the basis of IFN_{γ} expression is plotted on the Y axis.

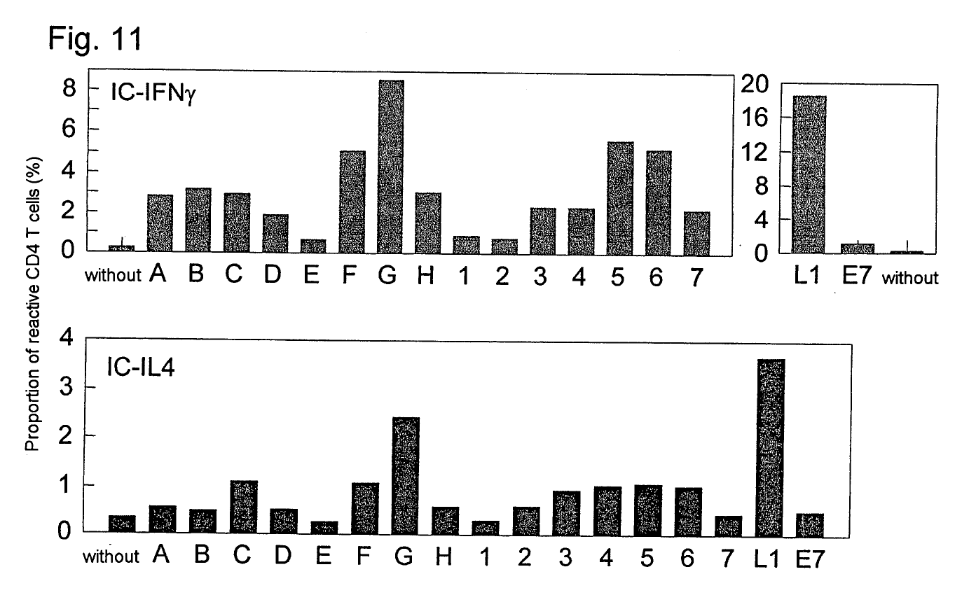

FIG. 11 shows the diagrammatic analysis of two FACScan experiments following restimulation of specific human T cells with donor-identical BLCL which are presenting different HPV18 peptide pools. The name of the respective peptide pool (A to H, 1 to 7) is given on the X axis; “without” stands for BLCL which were only incubated with buffer (negative control), while L1 and E7, respectively, stand for BLCL which were incubated with HPV18 L1 peptide pool and HPV18 E7 peptide pool, respectively (positive control). The proportion of CD4-positive T cells which were classified in the FACScan experiment as being reactive, and consequently as being T helper 1 cells (T_{H1}), on the basis of IFN_{γ} expression is plotted on the Y axis in the upper part of FIG. 11. The values for L1 and E7 were depicted, together with the negative control, in a separate graph using a different Y axis scale (upper right). The proportion of CD4-positive T cells which were classified in the FACScan experiment as being T helper 2 cells (T_{H2}) on the basis of IL-4 expression is plotted on the Y axis in the lower part of FIG. 11.

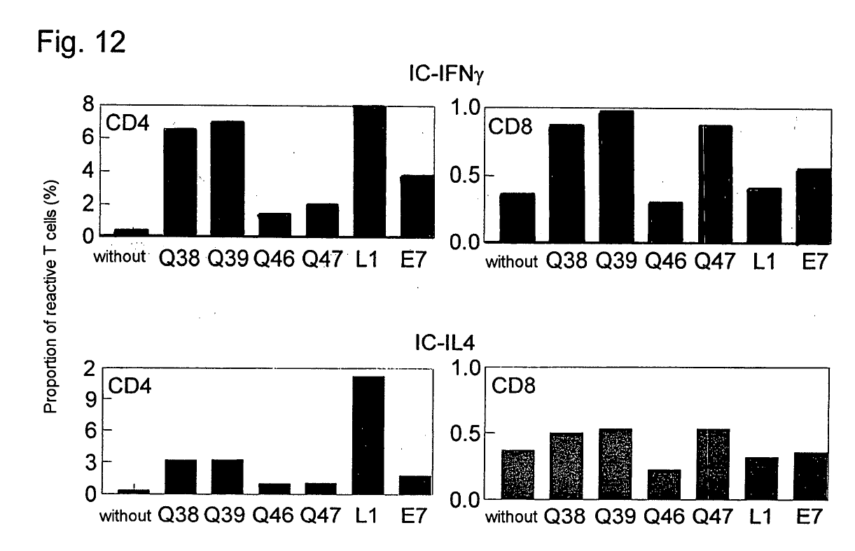

FIG. 12 shows the diagrammatic analysis of four FACScan experiments following restimulation of specific human T cells with donor-identical BLCL which are presenting different HPV18 peptides or peptide pools. The name of the respective peptide (Q38, Q39, Q46 and Q47) is given on the X axis; “without” stands for BLCL which were only incubated with buffer (negative control), while L1 and E7, respectively, stand for BLCL which were incubated with HPV18 L1 peptide pool and HPV18 E7 peptide pool, respectively (positive control). The proportion of reactive T cells in each case is given on the Y axis.

FIG. 12 (upper left) plots the proportion of CD4-positive T cells which were classified in the FACScan experiment as being reactive, and consequently as being T helper 1 cells (T_{H1}), on the basis of IFN_{γ} expression.

FIG. 12 (upper right) plots the proportion of CD8-positive T cells which were classified in the FACScan experiment as being reactive cytotoxic T cells on the basis of IFN_{γ} expression.

FIG. 12 (lower left) plots the proportion of CD4-positive T cells which were classified in the FACScan experiment as being T helper 2 cells (T_{H2}) on the basis of IL-4 expression.

FIG. 12 (lower right) plots the proportion of CD8-positive T cells which are expressing IL-4.

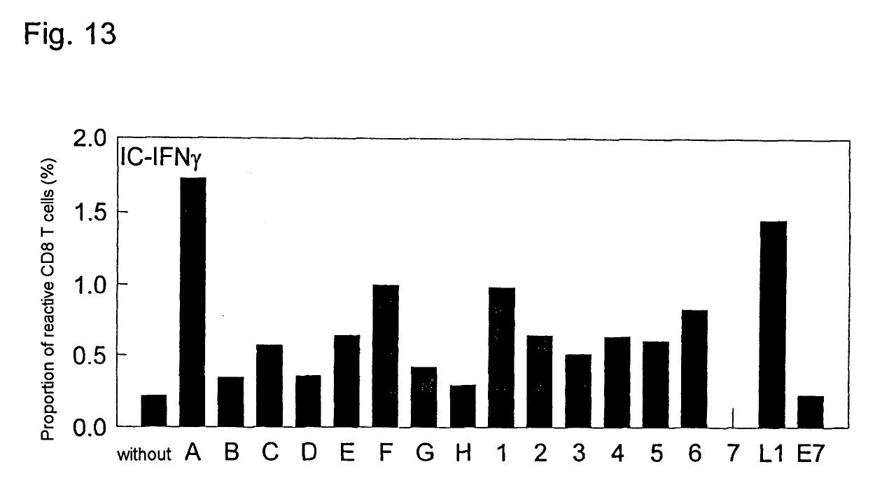

FIG. 13 shows the diagrammatic analysis of a FACScan experiment following restimulation of specific human T cells with donor-identical BLCL which are presenting different HPV16 peptide pools. The name of the respective peptide pool (A to H, 1 to 7) is given on the X axis; “without” stands for BLCL which were only incubated with buffer (negative control), while L1 and E7, respectively, stand for BLCL which were incubated with HPV16 L1 peptide pool and HPV16 E7 peptide pool, respectively (positive control). The proportion of CD8-positive T cells which were classified in the FACScan experiment as being reactive, and consequently as being cytotoxic T cells, on the basis of IFN_{γ} expression is plotted on the Y axis.

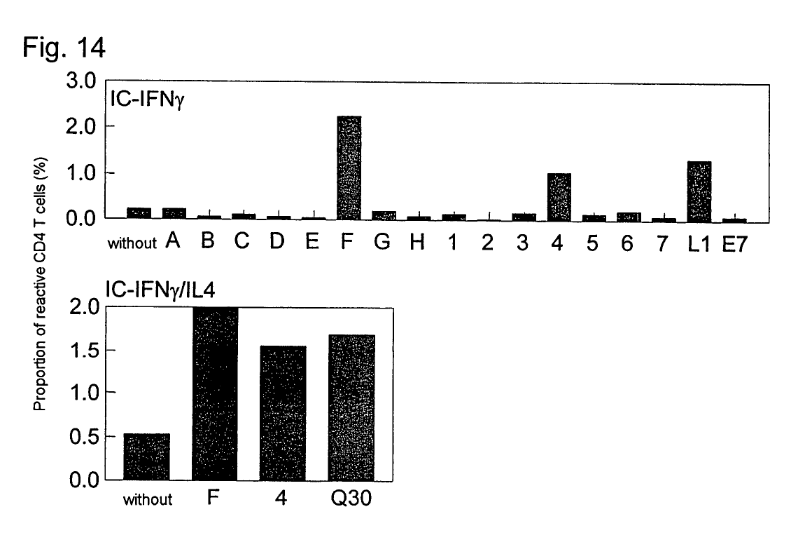

FIG. 14 shows the diagrammatic analysis of two FACScan experiments following restimulation of specific human T cells with donor-identical BLCL which are presenting different HPV18 peptide pools or a peptide. The name of the respective peptide pool (A to H, 1 to 7) or of the peptide Q30 is given on the X axis; “without” stands for BLCL which were only incubated with buffer (negative control), while L1 and E7, respectively, stand for BLCL which were incubated with HPV18 L1 peptide pool and HPV18 E7 peptide pool, respectively (positive control). The proportion of CD4-positive T cells which were classified in the FACScan experiment as being reactive, and consequently as being T helper 1 cells (T_{H1}), is plotted on the Y axis in the upper part of FIG. 14. The proportion of CD4-positive T cells which were classified in the FACScan experiment as being T helper 2 cells (T_{H2}) on the basis of IL-4 expression is plotted on the Y axis in the lower part of FIG. 14.

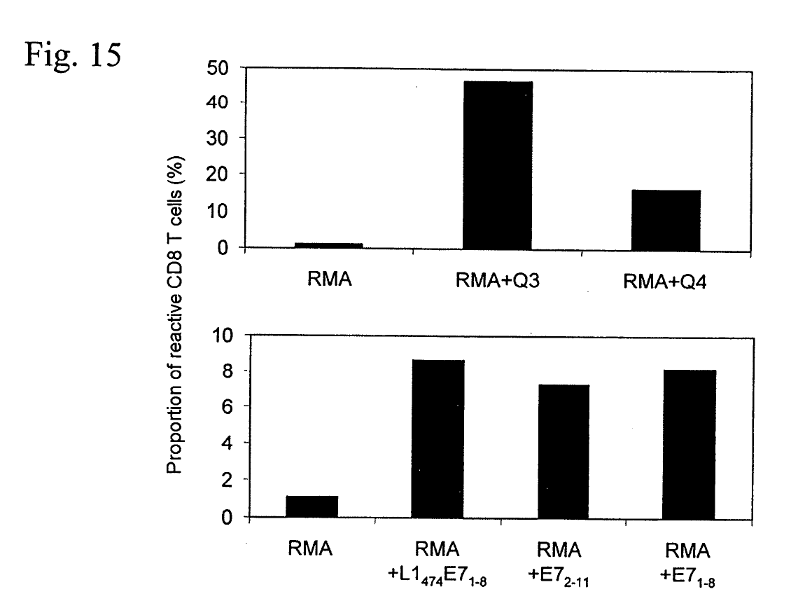

FIG. 15 shows the diagrammatic analysis of two FACScan experiments following restimulation of specific murine T cells with RMA cells which are presenting different peptides. The name of the respective peptide is given on the X axis; RMA cells without peptide were only incubated with buffer and served as a negative control. The proportion of CD8-positive T cells which were classified in the FACScan experiment as being reactive on the basis of IFN_{γ} expression is plotted on the Y axis.

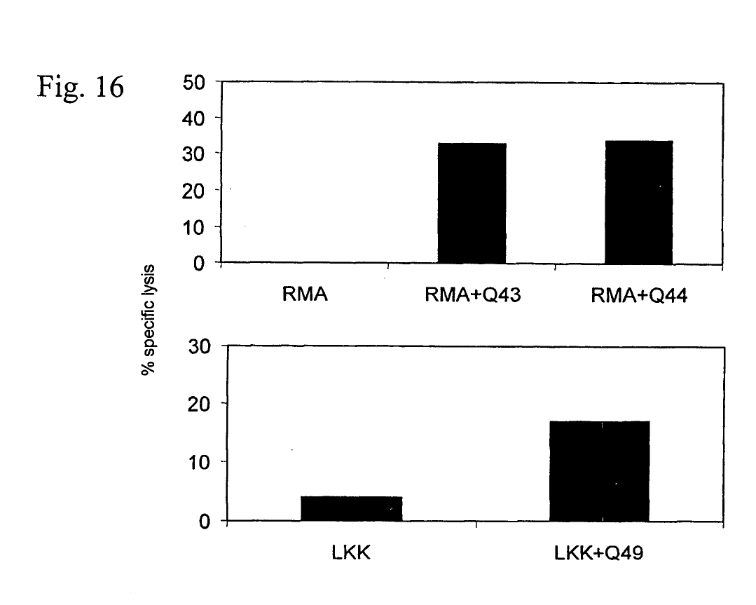

FIG. 16 shows the diagrammatic analysis of a ⁵¹Cr release experiment following the loading of RMA cells (top) and LKK cells (bottom), respectively, with the peptides Q43 and Q44 and, respectively, Q49 (target cells). The target cells were lysed by the T cells (effector cells) stimulated with the HPV18 L1 and E7 peptide pools, with the ratio of the effector cells employed to the target cells employed being 20. The cell type and the peptide are given on the X axis, with the cell without peptide functioning as a negative control. The % of the target cells which were specifically lysed, as determined by the release of ⁵¹Cr from the target cells, is plotted on the Y axis. The % values were calculated using the formula given in example 4.

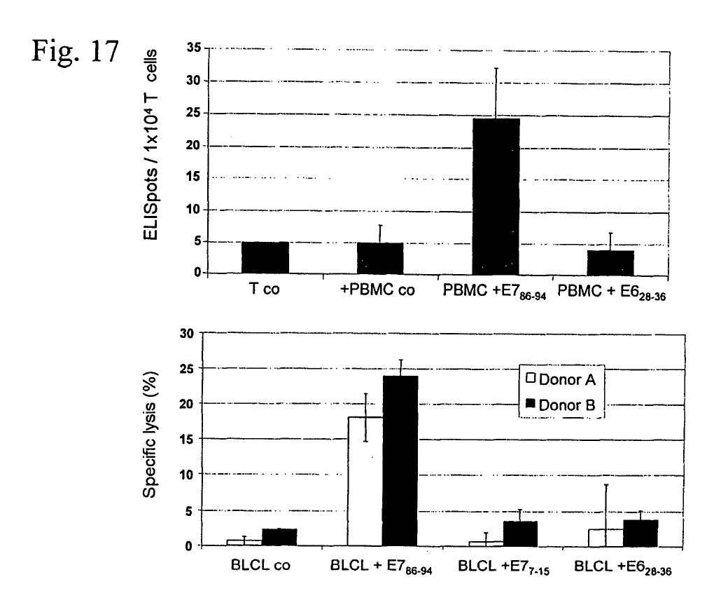

FIG. 17 top: HPV18 E7₈₆₋₉₄ epitope-reactive T cells in H7.1-A2-specific. T cell lines. A T cell line was generated by vaccinating in vitro with H7.1-A2 cells. The T cell line was tested in an ELISpot assay against autologous PBMC which were loaded with the peptide HPV18 E7₈₆₋₉₄. T co stands for a negative control using nonspecific T cells, while PBMC co stands for a negative control using PBMC which were not loaded with peptide.

FIG. 17 bottom: Cytolytic activitv of HPV18 E7₈₆₋₉₄-specific T cells. T cell lines directed against the epitope HPV18 E7₈₆₋₉₄ were prepared from two healthy blood donors and tested for the lysis of peptide-pulsed autologous BLCL in a cytotoxicity test (⁵¹chromium-release assay) (effector: target cell ratio of 30:1). BLCL co stands for a negative control of BLCL which were not pulsed with peptide.

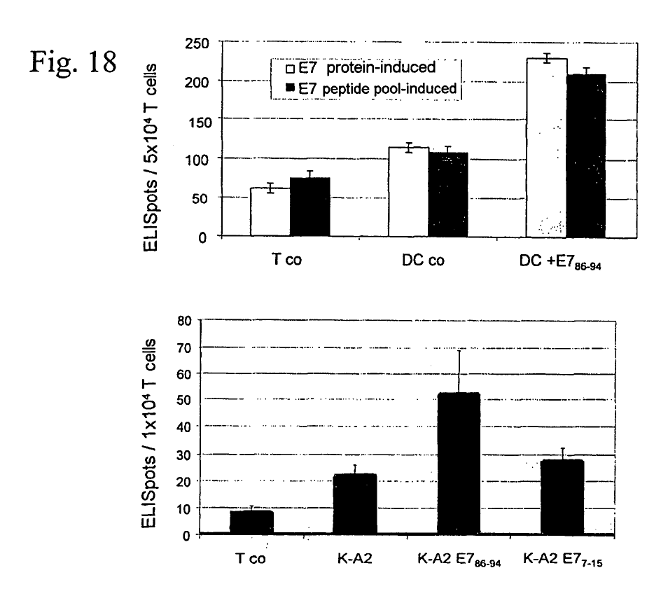

FIG. 18 top: Natural processing of the epitope HPV18 E7₈₆₋₉₄ by dendritic cells. T cell lines directed against HPV18 E7 were generated by vaccinating in vitro with protein-loaded or peptide pool-loaded autologous dendritic cells. The stimulation of HPV18 E7₈₆₋₉₄-specific T cells by epitope-loaded dendritic cells (DC) was measured in an ELISpot assay. T co stands for a negative control using nonspecific T cells, while DC co stands for a negative control of DCs which were not loaded with peptide.

FIG. 18 bottom: Presence of HPV18 E7₈₆₋₉₄-specific T cells in populations of tumor-infiltrating lymphocytes. TIL derived from an HPV18-positive and HLA-A2-positive female patient were expanded in vitro and tested in an ELISpot assay for reactivity with the HPV18 E7₈₆₋₉₄ epitope. T co stands for a negative control using nonspecific T cells while K-A2 stands for a negative control of K-A2 cells which were not loaded with peptide.

### EXAMPLES

### 1. Description of the Starting Materials

The HPV16L1_{ΔC*}E7₁₋₅₅ CVLPs were prepared as described in German patent application DE 19812941, see also Müller M. et al. (1997). Virology 234, 93-111.

PBMC denotes peripheral blood mononuclear cells, whose isolation is described, for example, in Rudolf M. P. et al. (1999), Biol. Chem. 380, 335-40.

BLCL denotes B cell lines which were in each case transformed with Epstein-Barr virus and which were prepared individually in the case of each blood donor (obtained from Dr Andreas Kaufmann, Jena, Germany).

CVLP-stimulated murine T cells were obtained as follows:

354 several C57BL/6 mice or C₃H mice were immunized twice with in each case 20 μg of HPV16L1_{ΔC*}E7₁₋₅₅ CVLPs or with a 1:1 mixture composed of HPV18 L1_{ΔCDI}E7_{1-53DI} CVLPs and HPV18L1_{ΔCDI}E7_{1-60DI} CVLPs, or with buffer in the case of the control. After 2 weeks, the spleen cells were isolated using standard methods.

Splenocytes were obtained as follows:

The spleen was removed from noninoculated mice and the spleen cells were resuspended using standard methods.

In connection with cells, APC stands for antigen-presenting cells.

JAWS cells were obtained from ATCC (CRL-11904).

LKK were obtained from ATCC (CCL-1).

RMA cells are derived from a thymoma in a C57BL/6 mouse (see Ljunggren H. U. and Kärre K. (1985) J. Exp. Med. 162, 1745-59).

α-Mouse CD8/PE denotes. a rat monoclonal antibody which is directed against the extracellular moiety of murine CD8 and contains the fluorescent label phycoerythrin (Pharmingen, Heidelberg, Germany).

α-Mouse CD4/cychrome denotes a rat monoclonal antibody which is directed against the extracellular moiety of murine CD4 and contains the fluorescent label cychrome (Pharmingen, Heidelberg, Germany).

α-Mouse IFN_{γ}/FITC denotes a rat monoclonal antibody which is directed against murine interferon y and which contains the fluorescent label FITC (Caltag, Hamburg, Germany).

α-Human CD8/APC denotes a mouse monoclonal antibody which is directed against the extracellular moiety of human CD8 and contains the fluorescent label APC (Caltag, Hamburg, Germany).

α-Human CD4/PerCP denotes a mouse monoclonal antibody which is directed against the extracellular moiety of human CD4 and contains the fluorescent label PerCP (Becton Dickinson, Hamburg, Germany).

α-Human IFN_{γ}/FITC denotes a mouse monoclonal antibody which is directed against human interferon γ and contains the fluorescent label FITC (Caltag, Hamburg, Germany).

α-Human IL4/PE denotes a mouse monoclonal antibody which is directed against human interleukin 4 and which contains the fluorescent label phycoerythrin (Caltag, Hamburg, Germany).

Human GM-CSF (Leukomax) was obtained from Novartis Pharma GmbH (Nurnberg, Germany).

Human IL4 was obtained from Becton Dickinson (Hamburg, Germany).

Human IL2 was obtained from Becton Dickinson (Hamburg, Germany).

Monensin was obtained from Sigma (Deisenhofen, Germany).

Triton X100 was obtained from Sigma (Deisenhofen, Germany).

Cells were in each case cultured at 37° C. and 5% CO₂ in RPMI medium (Gibco BRL, Eggenstein, Germany) containing 10% fetal calf serum, kanamycin and ampicillin.

Luma plates and the Canberra-Packerd B-Plate Counter were obtained from Canberra-Packerd, Dreieich, Germany.

FACScan Calibur denotes ‘fluorescence activated cell sorter’; the apparatus was obtained from Becton Dickenson (Hamburg, Germany).

Cellquest software was obtained from Becton Dickenson (Hamburg, Germany).

20 mer peptides which in each case overlapped by 9 amino acids, and which covered the sequences of the HPV16 L1 and E7 proteins, were synthesized. The peptides were numbered consecutively from 1 to 52. Their names and their sequences are compiled in the following table.

TABLE 1
Synthetic overlapping 20 mer peptides of HPV16
L1 and E7

| | | |
|---|---|---|
| Peptide | | relative |
| No. | Sequence | position |

HPV16 L1 peptides

| | | |
|---|---|---|
| P1 | MSLWLPSEATVYLPPVPVSK | 1-20 |
| P2 | YLPPVPVSKVVSTDEYVART | 12-31 |
| P3 | STDEYVARTNIYYHAGTSRL | 23-42 |
| P4 | YYHAGTSRLLAVGHPYFPIK | 34-53 |
| P5 | VGHPYFPIKKPNNNKILVPK | 45-64 |
| P6 | NNNKILVPKVSGLQYRVFRI | 56-75 |
| P7 | GLQYRVFRIHLPDPNKFGFP | 67-86 |
| P8 | PDPNKFGFPDTSFYNPDTQR | 78-97 |
| P9 | SFYNPDTQRLVWACVGVEVG | 89-108 |
| P10 | WACVGVEVGRGQPLGVGISG | 100-119 |
| P11 | QPLGVGISGHPLLNKLDDTE | 111-130 |
| P12 | LLNKLDDTENASAYAANAGV | 122-141 |
| P13 | SAYAANAGVDNRECISMDYK | 133-152 |
| P14 | RECISMDYKQTQLCLIGCKP | 144-163 |
| P15 | QLCLIGCKPPIGEHWGKGSP | 155-174 |
| P16 | GEHWGKGSPCTNVAVNPGDC | 166-185 |
| P17 | NVAVNPGDCPPLELINTVIQ | 177-196 |
| P18 | LELINTVIQDGDMVDTGFGA | 188-207 |
| P19 | DMVDTGFGAMDFTTLQANKS | 199-218 |
| P20 | FTTLQANKSEVPLDICTSIC | 210-229 |
| P21 | PLDICTSICKYPDYIKMVSE | 221-240 |
| P22 | PDYIKMVSEPYGDSLFFYLR | 232-251 |
| P23 | GDSLFFYLRREQMFVRHLFN | 243-262 |
| P24 | QMFVRHLFNPAGAVGENVPD | 254-273 |
| P25 | GAVGENVPDDLYIKGSGSTA | 265-284 |
| P26 | YIKGSGSTANLASSNYFPTP | 276-295 |
| P27 | ASSNYFPTPSGSMVTSDAQI | 287-306 |
| P28 | SMVTSDAQIFNKPYWLQRAQ | 298-317 |
| P29 | KPYWLQRAQGHNNGICWGNQ | 309-328 |
| P30 | NNGICWGNQLFVTVVDTTRS | 320-339 |
| P31 | VTVVDTTRSTNNSLCAAIST | 331-350 |
| P32 | MSLCAAISTSETTYKNTNFK | 342-361 |
| P33 | TTYKNTNFKEYLRHGEEYDL | 353-372 |
| P34 | LRHGEEYDLQFIFQLCKITL | 364-383 |
| P35 | IFQLCKITLTADVMTYIHSM | 375-394 |
| P36 | DVMTYIHSMNSTILEDWNFG | 386-405 |
| P37 | TILEDWNFGLQPPPGGTLED | 397-416 |
| P38 | PPPGGTLEDTYRFVTSQAIA | 408-427 |
| P39 | RFVTSQAIACQKHTPPAPKE | 419-438 |
| P40 | KHTPPAPKEDPLKKYTFWEV | 430-449 |
| P41 | LKKYTFWEVNLKEKFSADLD | 441-460 |
| P42 | KEKFSADLDQFPLGRKFLLQ | 452-471 |
| P43 | PLGRKFLLQAGMHGDTPTLH | 463-473 |
| | | and E7 |
| | | 1-9 |

HPV16 E7 peptides

| | | |
|---|---|---|
| P44 | MHGDTPTLHEYMLDLQPETT | 1-20 |
| P45 | MLDLQPETTDLYCYEQLNDS | 12-31 |
| P46 | YCYEQLNDSSEEEDEIDGPA | 23-42 |
| P47 | EEDEIDGPAGQAEPDRAHYN | 34-53 |
| P48 | AEPDRAHYNIVTFCCKCDST | 45-64 |
| P49 | TFCCKCDSTLRLCVQSTHVD | 56-75 |
| P50 | LCVQSTHVDIRTLEDLLMGT | 67-86 |
| P51 | TLEDLLMGTLGIVCPICSQKP | 78-97 |

“HPV16 L1 peptide pools” is understood as meaning the mixture of peptides P1 to P43 while “HPV16 E7 peptide pools” is understood as meaning the mixture of peptides P44 to P51.

20 mer peptides which covered the sequences of the HPV18 L1 and E7 proteins, and which in each case overlapped by 9 amino acids, were also synthesized. The peptides were consecutively numbered from 1 to 52. Their names and sequences are compiled in the following table.

TABLE 2
Synthetic overlapping 20 mer peptides of HPV18
L1 and E7

| | | |
|---|---|---|
| Peptide | | relative |
| No. | Sequence | position |

HPV18 L1 Peptide

| | | |
|---|---|---|
| Q1 | MALWRPSDNTVYLPPPSVAR | 1-20 |
| Q2 | YLPPPSVARVVNTDDYVTRT | 12-31 |
| Q3 | NTDDYVTRTSIFYHAGSSRL | 23-42 |
| Q4 | FYHAGSSRLLTVGNPYFRVP | 34-53 |
| Q5 | VGNPYFRVPAGGGNKQDIPK | 45-64 |
| Q6 | GGNKQDIPKVSAYQYRVFRV | 56-75 |
| Q7 | AYQYRVFRVQLPDPNKFGLP | 67-86 |
| Q8 | PDPNKFGLPDTSIYNPETQR | 78-97 |
| Q9 | SIYNPETQRLVWACAGVEIG | 89-108 |
| Q10 | WACAGVEIGRGQPLGVGLSG | 100-119 |
| Q11 | QPLGVGLSGHPFYNKLDDTE | 111-130 |
| Q12 | FYNKLDDTESSHAATSNVSE | 122-141 |
| Q13 | HAATSNVSEDVRDNVSVDYK | 133-152 |
| Q14 | RDNVSVDYKQTQLCILGCAP | 144-163 |
| Q15 | QLCILGCAPAIGEHWAKGTA | 155-174 |
| Q16 | GEHWAKGTACKSRPLSQGDC | 166-185 |
| Q17 | SRPLSQGDCPPLELKNTVLE | 177-196 |
| Q18 | LELKNTVLEDGDMVDTGYGA | 188-207 |
| Q19 | DMVDTGYGAMDFSTLQDTKC | 199-218 |
| Q20 | FSTLQDTKCEVPLDICQSIC | 210-229 |
| Q21 | PLDICQSICKYPDYLQMSAD | 221-240 |
| Q22 | PDYLQMSADPYGDSMFFCLR | 232-251 |
| Q23 | GDSMFFCLRREQLFARHFWN | 243-262 |
| Q24 | QLFARHFWNRAGTMGDTVPQ | 254-273 |
| Q25 | GTMGDTVPQSLYIKGTGMRA | 265-284 |
| Q26 | YIKGTGMRASPGSCVYSPSP | 276-295 |
| Q27 | GSCVYSPSPSGSIVTSDSQL | 287-306 |
| Q28 | SIVTSDSQLFNKPYWLHKAQ | 298-317 |
| Q29 | KPYWLHKAQGHNNGVCWHNQ | 309-328 |
| Q30 | NNGVCWHNQLFVTVVDTTRS | 320-339 |
| Q31 | VTVVDTTRSTNLTICASTQS | 331-350 |
| Q32 | LTICASTQSPVPGQYDATKF | 342-361 |
| Q33 | PGQYDATKFKQYSRHVEEYD | 353-372 |
| Q34 | YSRHVEEYDLQFIFQLCTIT | 364-383 |
| Q35 | FIFQLCTITLTADVMSYIHS | 375-394 |
| Q36 | ADVMSYIHSMNSSILEDWNF | 386-405 |
| Q37 | SSILEDWNFGVPPPPTTSLV | 397-416 |
| Q38 | PPPPTTSLVDTYRFVQSVAI | 408-427 |
| Q39 | YRFVQSVAITCQKDAAPAEN | 419-438 |
| Q40 | QKDAAPAENKDPYDKLKFWN | 430-449 |
| Q41 | PYDKLKFWNVDLKEKFSLDL | 441-460 |
| Q42 | LKEKFSLDLDQYPLGRKFLV | 452-471 |
| Q43 | YPLGRKFLVQAGMHGPKATL | 463-474 |
| | | and E7 |
| | | 1-8 |

HPV18 E7 Peptide

| | | |
|---|---|---|
| Q44 | MHGPKATLQDIVLHLEPQNE | 1-20 |
| Q45 | VLHLEPQNEIPVDLLCHEQL | 12-31 |
| Q46 | VDLLCHEQLSDSEEENDEID | 23-42 |
| Q47 | SEEENDEIDGVNHQHLPARR | 34-53 |
| Q48 | NHQHLPARRAEPQRHTMLCM | 45-64 |
| Q49 | PQRHTMLCMCCKCEARIKLV | 56-75 |
| Q50 | KCEARIKLVVESSADDLRAF | 67-86 |
| Q51 | SSADDLRAFQQLFLNTLSFV | 78-97 |
| Q52 | LFLNTLSFVCPWCASQQ | 89-104 |

“HPV18 L1 peptide pools” is understood as meaning the mixture of peptides Q1 to Q43 while “HPV18 E7 peptide pools” is understood as meaning the mixture of peptides Q44 to Q52.

### 2. Preparing HPV18 L1_{ΔCDI}E7_{1-53DI} CVLPs and HPV18 L1_{ΔCDI}E7_{1-6ODI} CVLPs

a) Preparing the Constructs

The nucleic acid encoding the individual papillomavirus-specific proteins were isolated from a gene library, for example by means of a PCR (“polymerase chain reaction”) amplification, and cloned. The HPV18 genome can be obtained universally under GenBank Accession No. X05015 and was published by Cole and Danos (J. Mol. Biol. 1987, 193 (4), 599-608).

In addition to this, the sequence which was used as the basis for constructing the fusion proteins according to the invention exhibited the following changes in the L1 gene: a C was replaced with a G, at the DNA level, at positions 89, 848, 1013 and 1230 in the L1 gene. At the protein level, the first three changes result in the replacement of Pro with Arg whereas the last mutation does not result in any change at the protein level. The E7 gene corresponds to the published sequence.

Another method of obtaining the desired nucleic acids is to use PCR to isolate the papillomavirus-specific genes directly from warts or tumors. Suitable primers for the HPV16 and HPV18 E6 and E7 genes are disclosed, for example, in WO93/21958. Examples of other literature references dealing with the desired nucleic acids are Kirnbaum, R. et al. (1994), J. Virol., 67, 6929-6936 or the clones deposited in the EMBL database which have already been mentioned above.

Two primers which are complementary to the HPV18L1 open reading frame (ORF) were constructed for preparing HPV18L1_{ΔCDI}. The first primer has the sequence

The first primer has the sequence
5′-ACC AGA CTC GAG ATG GCT TTG TGG CGG CCT AGT GAC-3′
while the second primer has the sequence
5′-ATA GCC AAG CTT AAT GAT ATC CTG AAC CAA AAA TTT ACG TCC-3′

The first primer encodes a XhoI restriction enzyme cleavage site 5′. The second primer encodes an EcoRV restriction enzyme cleavage site 5′. The EcoRV site is followed by a TAA translation stop codon in order to delete the last 35 amino acids of the HPV18L1 ORF. The PCR product was cleaved with XhoI/EcoRV and ligated into a pBluescript® vector which had also been cleaved with XhoI/EcoRV. The resulting construct, i.e. HPV18L1_{ΔCDI} pKS, was used in order to clone the HPV18E7_{1-53DI} and HPV18E7_{1-60DI} ORF's into the EcoRV site.

Primers possessing a 5′ EcoRV restriction enzyme cleavage site were used for cloning the HPV18 E7 fragments. The following primer pairs were employed:

5′-GGC CAT GAT ATC ATG CAT GGA CCT AAG GCA ACA

TTG-3′

(5′ end of the E7 gene)
and
5′-GGC CAT GAT ATC TCG TCG GGC TGG TAA ATG TTG

ATG-3′

(3′ end of the E7_{1-53DI} fragment)
or
5′-GGC CAT GAT ATC TGT GTG ACG TTG TGG TTC GGC

TC-3′

(3′ end of the E7_{1-60DI} fragment)

The PCR products were cleaved with EcoRV and inserted into the EcoRV site in the modified L1 gene.

The clones were analyzed by DNA sequencing. The HPV18L1_{ΔCDI}E7_{1-53DI} and HPV18L1_{ΔCDI}E7_{1-60DI} fusion genes, respectively, were then excised from the pBluescript® vector by means of BglII/EcoRI restriction digestion and ligated into the BglII/EcoRI-cleaved baculovirus transfer vector pVL1392 in order to prepare recombinant baculoviruses.

b) Preparing Recombinant Baculoviruses

Spodoptera frugiperda (Sf9) cells were and propagated as a monolayer or in suspension culture in TNM-FH insect medium (Life Technologies, Karlsruhe) containing 10% fetal calf serum. Recombinant baculoviruses were prepared by cotransfecting 5 μg of the recombinant plasmids and 1 μg of linearized Baculo-Gold® DNA (Pharmingen, San Diego, Calif.) into Sf9 cells. Recombinant viruses were purified by end point dilution and/or plaque isolation. In order to test expression, 10⁶ Sf9 cells were infected with recombinant baculovirus at m.o.i.'s (multiplicities of infection) of 0.5 and 1 for 48 h. After the incubation, the medium was removed and the cells were washed with PBS (140 mM NaCl, 2.7 mM KCl, 8.1 mM Na₂PO₄, 1.5 mM KH₂PO₄, pH 7.2). The cells were then analyzed by FACS measurement or lysed in SDS sample buffer and tested by SDS gel chromatography and immunoblot assay.

c) Purifying Chimeric Virus-like Particles

In order to prepare CVLPs, Sf9 or SF+ cells were cultured, at 27° C., in the serum-free media InsectXPress (Biowhittaker, Verviers, Belgium) or Sf 900II (Life Technologies, Karlsruhe) up to a density of 1.5-2×10⁶ cells per ml. A 200 ml culture was infected with recombinant baculoviruses for 48 h with an m.o.i. of from 1 to 2. The cells were then pelleted and frozen at −80° C. Freeze-thaw lysis then took place by adding 4 vol. of extraction buffer (200 mM NaCl, 50 mM Tris, pH 8.5). The homogenate was clarified by centrifuging at 10.000 rpm in a Sorvall SS34 rotor. The L1E7 protein was purified, for the immunological assays, from the clarified crude extract by means of precipitating with ammonium sulfate at 35-40% saturation and then performing anion exchange chromatography on Fractogel® TMAE (Merck, Darmstadt), with the CVLPs being eluted at 300-400 mM NaCl in a linear salt gradient. The protein contents of the purified fractions were determined by the Bradford method using bovine serum albumin as the standard.

### 3. Restimulating HPV16 L1 Peptide-stimulated Murine T Cells With Different Antigen-presenting Cells

Murine T cells (4×10⁵) derived from HPV16L1_{ΔC*}E7₁₋₅₅ CVLP-inoculated C57BL/6 mice were stimulated for 5 weeks with HPV16 L1 peptide pools at 37° C., with the weekly addition of 1 μg of each individual peptide/ml and 10⁵ antigen-presenting cells (irradiated splenocytes), and harvested. The cells were subsequently restimulated, in 100 μl of medium at 37° C., with 1 μg of the peptides given on the X axis in FIG. 1/ml and 10⁵ antigen-presenting cells (JAWS) in the presence of 10 IU of IL2/ml. Cells which were only incubated with buffer serve as the negative control.

After an hour, 1 μl of monensin (300 μM) was added. The cells were incubated at 37° C. for a further 5 hours. The cells were then fixed and permeabilized and stained with α-mouse CD8/PE, with α-mouse CD4/cychrome and with α-mouse interferon γ/FITC. The labeling of the cells was analyzed in a FACScan caliber and the results of the measurements were analyzed using Cellquest software.

Result: When incubated with peptides P18, P19 or P43, as shown in the upper part of FIG. 1, or with peptides P35 or P3, as shown in the lower part of FIG. 1, JAWS cells brought about a restimulation of peptide-stimulated CD8-positive murine T cells. Peptides P3, P18, P19, P35 and P43 consequently contain H2b-restricted cytotoxic T cell epitopes.

### 4. Lysing P33-loaded RMA Cells

RMA cells were incubated at 37° C. for one hour with ⁵¹Cr, washed three times with medium and divided into 2 aliquots. 10 μg of the P33 peptide/ml were added to one aliquot of the cells while the other aliquot served as the negative control in the absence of the peptide. In each case 2000 of the cells (=target cells) were then added to 40.000 T cells (=effector cells) in a total volume of 150 μl. The T cells had previously been stimulated for 5 weeks with a mixture composed of 43 peptides (peptides 1-43, in each case 1 μg/ml).

Assay mixtures for spontaneous and maximal lysis of the cells were set up in parallel. Target cells which were incubated with culture medium were used for the spontaneous lysis. For the maximal lysis, target cells were lysed by adding 0.5% Triton X100.

The assay mixtures were incubated at 37° C. for 5 h. 50 μl volumes of the supernatants from the assay mixtures were added to Luma plates and dried overnight at room temperature. On the following morning, the quantity of radioactive ⁵¹Cr (counts) was determined using a Canberra-Packerd B Plate Counter and related to the maximally lysed cells in the Triton assay mixture. The % specific lysis was then determined using the formula:

x=100·(counts−spontaneous counts)/(maximal counts−spontaneous counts).

FIG. 2 shows that, while the T cells were able to efficiently lyse the RMA cells which were loaded with the P33 peptide, they were unable to lyse the unloaded RMA cells. The P33 peptide is consequently an H2b-restricted cytotoxic T cell epitope.

### 5. Restimulating HPV18 Peptide-stimulated Murine T Cells With Different Antigen-presenting Cells

Murine T cells (4×10⁵) derived from C₃H mice inoculated with HPV18 L1_{ΔCDI}E7_{1-53DI} CVLPs and HPV18 L1_{ΔCDI}E7_{1-6ODI} CVLPs were stimulated for 5 weeks with HPV18 L1 or E7 peptide pools at 37° C., with the weekly addition of 1 μg of each individual peptide/ml and 10⁵ antigen-presenting cells (irradiated splenocytes), and harvested. The cells were then restimulated, at 37° C. and in 100 μl of medium, with 1 μg of the peptides given on the X axis in FIG. 3/ml and 10⁵ antigen-presenting cells (LKK) in the presence of 10 IU of IL2/ml. Cells which were only incubated with buffer served as the negative control.

1 μl of monensin (300 μm) was added after an hour. The cells were incubated at 37° C. for a further 5 hours. The cells were then fixed and permeabilized and stained with α-mouse CD8/PE, with α-mouse CD4/cychrome and with α-mouse interferony/FITC. The labeling of the cells was analyzed in a FACScan Calibur and the results of the measurement were analyzed using Cellquest software.

Result: When incubated with the peptides Q22, Q23, Q51, Q43 and Q44, as shown in FIG. 3, and with the peptides Q41 and Q5, as shown in FIG. 4, LKK cells brought about a restimulation of peptide-stimulated murine CD8-positive T cells. The peptides Q5, Q22, Q23, Q41, Q43, Q44 and Q51 consequently contain H2k-restricted cytotoxic T cell epitopes.

### 6. Restimulating HPV18 CVLP-stimulated T Cells With Different Antigen-presenting Cells

Human T cells (4×10⁵) from a non-HLA-typed blood donor were stimulated for 1 week with HPV18 L1_{ΔCDI}E7_{1-53DI} CVLPs and HPV18 L1_{ΔACDI}E7_{1-60DI} CVLPS, 800 U of human GM-CSF/ml and 500 U of human IL4/ml and also, for a further 5 weeks, with HPV18 L1_{ΔCDI}E7_{1-53DI} CVLPs and HPV18 L1_{ΔCDI}E7_{1-6ODI} CVLPs at 37° C., with the weekly addition of 1 μg of the CVLPs mixLure (ratio of the two constructs 1:1)/ml and 10⁵ antigen-presenting cells (irradiated PMBC), and harvested.

The 20 mer peptides Q1 to 52 were assembled into peptide pools A to H and 1 to 7, respectively, in accordance with the matrix

| HPV18 Pools | A | B | C | D | E | F | G | H |
|---|---|---|---|---|---|---|---|---|
| 1 | Q 1 | Q 2 | Q 3 | Q 4 | Q 5 | Q 6 | Q 7 | Q 8 |
| 2 | Q 9 | Q 10 | Q 11 | Q 12 | Q 13 | Q 14 | Q 15 | Q 16 |
| 3 | Q 17 | Q 18 | Q 19 | Q 20 | Q 21 | Q 22 | Q 23 | Q 24 |
| 4 | Q 25 | Q 26 | Q 27 | Q 28 | Q 29 | Q 30 | Q 31 | Q 32 |
| 5 | Q 33 | Q 34 | Q 35 | Q 36 | Q 37 | Q 38 | Q 39 | Q 40 |
| 6 | Q 41 | Q 42 | Q 43 | Q 44 | Q 45 | Q 46 | Q 47 | Q 48 |
| 7 | Q 49 | Q 50 | Q 51 | Q 52 | | | | |

The T cells were then restimulated, at 37° C. and in 100 μl of medium, with the peptide pools and 10⁵ antigen-presenting cells (donor-identical BLCL) in the presence of 10 IU of IL2/ml. The quantities of the peptide pools which were used in this connection were such that. 1 μg/ml was added in the case of each individual peptide.

Cells which were only incubated with buffer served as the negative control while cells incubated with the HPV18 L1 peptide pool and the HPV18 E7 peptide pool, respectively, served as positive controls.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was analyzed in a FACScan Calibur and the results of the measurements were analyzed using Cellquest software.

Result: FIG. 5 shows that the BLCL incubated with the peptide pools F and 1, in particular, brought about a restimulation of CVLP-stimulated human CD4-positive T cells. Furthermore, peptide pools A and 6 exhibited a restimulation which was clearly higher than that of the negative control. By contrast, the BLCL which were incubated with the other peptide pools, and the negative control or the BLCL which were incubated with the E7 peptide pool, only exhibited a small proportion of reactive CD4-positive T cells. The peptide pools F and 1 jointly contain the peptide Q6, which is consequently responsible for restimulating the CVLP-stimulated T cells. Peptide Q6 consequently contains a T helper epitope. Peptide pools A and 1 contain peptide Q1 as the peptide possessed in common, while this peptide is Q41 in the case of A and 6 and Q46 in the case of F and 6. Consequently, the peptides Q1, Q41 and Q46 also contain T helper epitopes.

### 7. Restimulating HPV16 CVLP-stimulated T Cells With Different Antigen-presenting Cells

In analogy with example 6, human T cells derived from a non-HLA-typed blood donor were stimulated with HPV16L1_{ΔC*}E7₁₋₅₅ CVLPs and harvested.

The 20mer peptides P1 to 51 were assembled into peptide pools A to H and 1 to 7, respectively, in accordance with the following matrix

| HPV16 Pools | A | B | C | D | E | F | G | H |
|---|---|---|---|---|---|---|---|---|
| 1 | P 1 | P 2 | P 3 | P 4 | P 5 | P 6 | P7 | P8 |
| 2 | P 9 | P 10 | P 11 | P 12 | P 13 | P 14 | P 15 | P 16 |
| 3 | P 17 | P 18 | P 19 | P 20 | P 21 | P 22 | P 23 | P 24 |
| 4 | P 25 | P 26 | P 27 | P 28 | P 29 | P 30 | P 31 | P 32 |
| 5 | P 33 | P 34 | P 35 | P 36 | P 37 | P 38 | P 39 | P 40 |
| 6 | P 41 | P 42 | P 43 | P 44 | P 45 | P 46 | P 47 | P 48 |
| 7 | P 49 | P 50 | P 51 | | | | | |

The T cells were then restimulated as described in example 6, fixed, permeabilized and stained. The analysis also took place as described in example 6.

Result: FIG. 6 shows that BLCL which were incubated with peptide pools G and 3 brought about a restimulation of CVLP-stimulated T cells. In addition, peptide pools B and C, and also 2 and 4, exhibited a restimulation which was clearly higher than that of the negative control. By contrast, the PBMC which were incubated with the other peptide pools, and the PBMC which were incubated as the negative control, only exhibited a small proportion of reactive CD4-positive T cells; however, the BLCL which were incubated with the other peptide pools, and the negative control and the PBMC which were incubated with the E7 peptide pool, did not do so. Peptide pools G and 3 contain peptide P23 in common, while B and 2 contain P10 in common, B and 3 contain P18 in common, B and 4 contain P26 in common, C and 2 contain P11 in common, C and 3 contain P19 in common, C and 4 contain P27 in common, G and 2 contain P15 in common, and G and 4 contain P31 in common. These peptides are consequently in each case responsible for restimulating the CVLP-stimulated T cells. Peptides P10, P11, P15, P18, P19, P23, P26, P27 and P31 consequently in each case contain a T helper epitope.

### 8. Restimulating HPV18 L1 Peptide-stimulated T Cells With Different Antigen-presenting Cells

In analogy with example 3, human T cells (4×10⁵) derived from an HLA A24-positive donor were stimulated for 3 weeks with the HPV18 L1 peptide pool, at 37° C., with the weekly addition of 1 μg of each individual peptide/ml and 10⁵ antigen-presenting cells (irradiated PMBC), and harvested.

The cells were then restimulated, at 37° C. in 100 μl of medium, with 10 μg of the HPV18 L1 peptide Q9/ml and 10⁵ antigen-presenting cells (donor-identical BLCL) in the presence of 10 IU of IL2/ml. Cells which were only incubated with buffer served as the negative control.

1 μl of monensin (300 μM) was added after an hour. The cells were then incubated at 37° C. for a further 5 hours. After that, the cells were then fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 7 shows that the BLCL cells which were incubated with peptide Q9 brought about a restimulation of HPV18 L1 peptide pool-stimulated CD8-positive T cells. Consequently, peptide Q9 contains an HLA A24-restricted cytotoxic T cell epitope.

Using the algorithm for potential HLA A24-binding peptides (Paker, KC et al. (1994) J. Immunol. 152:163), which was implemented in what is termed Parker's Peptide Prediction Program under http://www-bimas.dcrt.nih.gov/molbio/hla_bind/, it was established that the peptide IYNPETQRL binds to MHC class I molecules of the HLA A24 haplotype. The peptide IYNPETQRL is consequently responsible for the restimulation of the T cells by the BLCL cells which were incubated with the Q9 peptide.

Consequently, the peptide IYNPETQRL, which is contained in peptide Q9 of the overlapping 20mers, is an HLA A24-restricted cytotoxic T cell epitope.

### 9. Restimulating HPV16 CVLP-stimulated T Cells With Different Antigen-presenting Cells

In analogy with example 6, human T cells derived from a non-HLA-typed blood donor were stimulated with HPV16L1_{ΔC*}E7₁₋₅₅ CVLPs and harvested.

The T cells were then restimulated, at 37° C. and in 100 μl of medium, with the peptide pools from example 7 and 10⁵ antigen-presenting cells (donor-identical BLCL) in the presence of 10 IU of IL2/ml. Use was made, in this connection, of quantities of the peptide pools which was such that 1 μg/ml was added in the case of each individual peptide. Cells which were only incubated with buffer served as the negative control while cells which were incubated with HPV16 L1 peptide pool served as positive controls.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 8 (top) shows that the proportion of CD4-positive and IFN_{γ}-positive cells is very high in the case of the BLCL which were incubated with peptide pools G and 5 as well as in the case of the BLCL which were incubated with P39. Since peptide P39 is that which is contained in common in peptide pools G and 5 and which on its own likewise brought about a restimulation of CVLP-stimulated T cells, peptide P39 contains a T helper epitope. Since CD4-positive and IFN_{γ}-positive cells are as a rule T_{H1}, the epitope is consequently a T_{H1} epitope.

In addition, the proportion of CD4-positive and IL4-positive cells (lower part of FIG. 8) was markedly higher in the case of the BLCL incubated with peptide pools F, G and 5 than was the proportion of reactive cells in the case of the BLCL which were incubated with the other peptide pools or in the case of the negative control. As a rule, CD4-positive and IL4-positive cells are T_{H2} cells. This in turn means that peptide P39, contained in G and 5, also contains a T_{H2} epitope in addition, or is identical to the T_{H1} epitope, and that peptide 38, contained in F and 5, also contains a T_{H2} epitope.

### 10. Restimulating HPV16 CVLP-stimulated T Cells With Different Antigen-presenting Cells

In analogy with example 6, human T cells derived from a non-HLA-typed blood donor were stimulated with HPV16L1_{ΔC*}E7₁₋₅₅ CVLPs and harvested.

The T cells were then restimulated, at 37° C. and in 100 μl of medium, with the peptide pools from example 7 and 10⁵ antigen-presenting cells (donor-identical BLCL) in the presence of 10 IU of IL2/ml. Use was made, in this connection, of quantities of the peptide pools which was such that 1 μg/ml was added in the case of each individual peptide. Cells which were only incubated with buffer served as the negative control while cells which were incubated with HPV16 L1 peptide pool served as positive controls.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 9 shows that the proportion of CD8-positive T cells is high in the case of the BLCL which were incubated with peptide pools B, E, G and 1. Since peptide pools B and 1 contain peptide P2 in common, while peptide pools E and 1 contain P5 in common and peptide pools G and 1 contain P7 in common, peptides P2, P5 and P7 each contain a cytotoxic T cell epitope.

### 11. Restimulating HPV16 CVLP-stimulated T Cells With P33-stimulated Antigen-presenting Cells

In analogy with example 6, human T cells derived from a non-HLA-typed blood donor were stimulated with HPV16L1_{ΔC*}E7₁₋₅₅ CVLPs and harvested.

The T cells were then restimulated, at 37° C. and in 100 μl of medium, with 1 μg of peptide P33/ml and 10⁵ antigen-presenting cells (donor-identical BLCL) in the presence of 10 IU of IL2/ml. Cells which were only incubated with buffer served as the negative control while cells which were incubated with HPV16 L1 peptide pool served as the positive control.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 10 shows that the proportion of CD8-positive T cells is markedly higher in the case of the BLCL which were incubated with the P33 peptide than in the case of the negative control. P33 consequently contains a cytotoxic T cell epitope.

### 12. Restimulating HPV18 CVLP-stimulated T Cells With Different Antigen-presenting Cells

In analogy with example 6, human T cells derived from a non-HLA-typed blood donor were stimulated, at 37° C., with HPV18L1_{ΔCDI}E7_{1-53DI} CVLPs and HPV18L1_{ΔCDI}E7_{1-60DI} CVLPs and harvested.

The T cells were then restimulated, at 37° C. and in 100 μl of medium, with the HPV18 peptide pools from example 6 and 10⁵ antigen-presenting cells (donor-identical BLCL) in the presence of 10 IU of IL2/ml. Use was made, in this connection, of quantities of the peptide pools which were such that 1 μg/ml was added in the case of each individual peptide. Cells which were only incubated with buffer served as the negative control while cells which were incubated with HPV18 L1 peptide pool served as the positive control.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 11 (top) shows that the proportion of CD4-positive and IFN_{γ}-positive cells is high in the case of the BLCL which were incubated with peptide pools F, G, 5 and 6. Peptide pools F and 5 possess peptide Q38 in common, while peptide pools F and 6 possess peptide Q46 in common, peptide pools G and 5 possess peptide Q39 in common and peptide pools G and 6 possess peptide Q47 in common. Consequently, peptides Q38, Q39, Q46 and Q47 each contain a T_{H1} epitope.

FIG. 11 (bottom) shows that the proportion of CD4-positive and IL4-positive cells is particularly high in the case of peptide pool G; however, the proportion of reactive cells are about equally high in the case of pools 3, 4, 5 and 6, meaning that it was not possible to draw any unambiguous conclusion as regards the T_{H2} epitopes from this experiment. However, peptides Q38, Q39, Q46 and Q47 were tested individually in the following example.

### 13. Restimulating HPV18 CVLP-stimulated T Cells With Antigen-presenting Cells Which Were Stimulated With Q38 Peptide, Q39 Peptide, Q46 Peptide or Q47 Peptide

In analogy with example 6, human T cells (4×10⁵) derived from a non-HLA-typed blood donor were stimulated with HPV18L1_{ΔCDI}E7_{1-53DI} CVLPs and HPV18L1_{ΔCDI}E7_{1-60DI} CVLPs and harvested.

The T cells were then restimulated, at 37° C. and in 100 μl of medium, with in each case 1 μg of peptide Q38, Q39, Q46 or Q47/ml and 10⁵ antigen-presenting cells (donor-identical BLCL) in the presence of 10 IU of IL2/ml. Cells which were only incubated with buffer served as the negative control while cells which were incubated with the HPV18 L1 peptide pool or the E7 peptide pool served as positive controls.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 12 (top left) shows that, while the proportion of reactive CD4-positive and IFN_{γ}-positive T cells is particularly high in the case of the BLCL which were incubated with peptides Q38 and Q39, it is also still markedly higher than in the negative control in the case of peptides Q46 and Q47. Consequently, Q38, Q39, Q46 and Q47 each contain a T_{H1} epitope.

FIG. 12 (bottom left) shows that the proportion of reactive CD4-positive and IL4-positive T cells is particularly high in the case of peptides Q38 and Q39, meaning that, in addition to the T_{H1} epitopes, Q38 and Q39 also contain T_{H2} epitopes in addition or that the T_{H1} epitopes are also at the same time T_{H2} epitopes.

FIG. 12 (top right) shows that the proportion of reactive CD8-positive and IFN_{γ}-positive T cells is particularly high in the case of peptides Q38, Q39 and Q47, meaning that peptides Q38, Q39 and Q47 additionally contain cytotoxic T cell epitopes. The proportion of CD8-positive and IL4-positive cells is low, and comparable to the negative control, in the case of peptides Q38, Q39, Q46 and Q47, a finding which can be explained by the fact that CD8-positive cells do not as a rule express any IL4.

### 14. Restimulating HPV16 CVLP-stimulated T Cells With Different Antigen-presenting Cells

In analogy with example 6, human T cells derived from a non-HLA-typed blood donor were stimulated with HPV16L1_{ΔC*}E7₁₋₅₅ CVLPs and harvested.

The T cells were then restimulated, at 37° C. and in 100 μl of medium, with the peptide pools from example 7 and 10⁵ antigen-presenting cells (donor-identical BLCL) in the presence of 10 IU of IL2/ml. Use was made, in this connection, of quantities of the peptide pools which were such that 1 μg/ml was added in the case of each individual peptide. Cells which were only incubated with buffer served as the negative control, while cells which were incubated with the HPV16 L1 or E7 peptide pool served as positive controls.

1 μl of monensin was added after an hour. The cells were then incubated at 37° C. for a further 5 hours. After that, the cells were fixed and permeabilized and stained with α-human CD8/APC, with α-human CD4/PerCP and with α-human IFN_{γ}/FITC. The labeling of the cells was examined in a FACScan Calibur and the results of the measurements were analyzed using Cellquest software.

Result: FIG. 13 shows that the proportion of CD8-positive and IFN_{γ}-positive T cells is high in the case of the BLCL which were incubated with peptide pools A, C, E, F, 1 and 6. Peptide pools A and 1 contain peptide P1 in common, while peptide pools C and 1 contain peptide P3 in common, peptide pools E and 1 contain peptide P5 in common, peptide pools F and 1 contain peptide P6 in common, peptide pools A and 6 contain peptide P41 in common, peptide pools C and 6 contain peptide P43 in common, peptide pools E and 6 contain peptide P45 in common and peptide pools F and 6 contain peptide P46 in common. Consequently, peptides P1, P3, P5, P6, P41, P43, P45 and P46 each contain a cytotoxic T cell epitope.

### 15. Restimulating HPV18 CVLP-stimulated T Cells With Different Antigen-presenting Cells

In analogy with example 6, human T cells derived from a non-HLA-typed blood donor were stimulated, at 37° C., with HPV18L1_{ΔCDI}E7_{1-53DI} CVLPs and HPV18L1_{ΔCDI}E7_{1-60DI} CVLPs and harvested.

The T cells were then restimulated, at 37° C. and in 100 μl of medium, with the peptide pools from example 6 and 10⁵ antigen-presenting cells (donor-identical BLCL) in the presence of 10 IU of IL2/ml. Use was made, in this connection, of quantities of the peptide pools which were such that 1 μg/ml was added in the case of each individual peptide. Cells which were only incubated with buffer served as the negative control while cells which were incubated with the HPV18 L1 or E7 peptide pool served as positive controls (upper part of FIG. 14). In the case of the lower part of FIG. 14, the T cells were restimulated, at 37° C. and in 100 μl of medium, with peptide pools F and 4 and with 1 μg of peptide Q30/ml and 10⁵ antigen-presenting cells (donor-identical BLCL) in the presence of 10 IU of IL2/ml.

1 μl of monensin was added after an hour. The cells were then incubated at 37° C. for a further 5 hours. After that, the cells were fixed and permeabilized and stained with α-human CDB/APC, with α-human CD4/PerCP and with α-human IFN_{γ}/FITC. The labeling of the cells was examined in a FACScan Calibur and the results of the measurements were analyzed using Cellquest software.

Result: FIG. 14 (top) shows that the proportion of CD4-positive and IFN_{γ}-positive T cells is high in the case of the BLCL which were incubated with peptide pools F and 4. Peptide pools F and 4 contain peptide Q30 in common. Consequently, peptide Q30 contains a T_{H1} epitope.

This result was confirmed by the experiment depicted in FIG. 14. The proportion of reactive CD4-positive and IFN_{γ}-positive T cells is in each case comparable, irrespective of whether the T cells were restimulated with BLCL which were incubated with peptide pools F or 4 or with the peptide Q30.

### 16. Restimulating HPV18 Peptide-stimulated Murine T Cells

In analogy with example 5, murine T cells derived from CVLP-inoculated mice were stimulated with L1 or E7 peptide pools in the added presence of antigen-presenting cells. The cells were then restimulated with the peptides given on the X axis in FIG. 15 (Q3 and Q4 in the upper part of FIG. 15 and L1₄₇₄E7₁₋₈, E7₂₋₁₁ and E7₁₋₈ in the lower part of FIG. 15) and also antigen-presenting cells (RMA). Cells which were only incubated with buffer served as the negative control.

The experiment was also analyzed in analogy with example 5, with the reactive CD8-positive cells being determined by means of antibody staining and FACScan.

Result: FIG. 15 shows that, when incubated with peptides Q3 and Q4 (upper part of figure) and peptides L1₄₇₄E7₁₋₈, E7₂₋₁₁ and E7₁₋₈ (lower part of figure), RMA cells brought about a restimulation of peptide-stimulated murine CD8 T cells. Peptides Q3, Q4, L1₄₇₄E7₁₋₈, E7₂₋₁₁ and E7₁₋₈ consequently each contain an H2b-restricted cytotoxic T cell epitope.

### 17. Lysing HPV18 Peptide-loaded RMA Cells/LKK Cells

In analogy with example 4, RMA or LKK cells were radioactively labeled with ⁵¹Cr and incubated with the peptides (Q43 and Q44 in the upper part of FIG. 16 and Q49 in the lower part of FIG. 16) which are plotted on the X axis. Cells without peptide served as negative controls. T cells which had previously been stimulated with the peptide pool were then added.

The lysis of the RMA cells or LKK cells by the T cells was measured by the release of the ⁵¹Cr and calculated as described in example 4.

Result: FIG. 16 shows that, while the T cells were able to efficiently lyse the RMA cells which were loaded with Q43 or Q44 (upper part of FIG. 16) or the LKK cells which were loaded with Q49 (lower part of FIG. 16), they were unable to lyse the unloaded RMA cells or LKK cells. The Q43 peptide and the Q44 peptide are consequently H2b-restricted cytotoxic T cell epitopes while Q49 is consequently an H2k-restricted cytotoxic T cell epitope.

### 18. Identifying the Cytolytic T Cell Epitope HPV18 E7₈₆₋₉₄

A HLA Binding Prediction

The NIH HLA binding prediction program (address: http://www-bimas.dcrt.nih.gov/molbio/hla\_bind/) was used for predicting 9 mer sequences in the HPV type 18 E7 protein which would bind with high strength to HLA A*0201. 9 mers were examined since they represent the optimal ligands for HLA class I molecules. For the prediction, the complete amino acid sequence of the HPV18 E7 protein was fed into the program and it was stipulated that 9 mers for the HLA A*0201 allele should be predicted. The program supplies a list in which the 9 mers which have been found are arranged in accordance with binding strength. The value for the binding strength is given as the half-change value in minutes for the dissociation of a peptide having the corresponding amino acid sequence (T_{1/2}).

B In Vitro Vaccination of PBMC

The predicted peptides were synthesized using standard methods (F moc method). Peptide-specific T cell lines were established against peptides which had a high predicted strength of binding to HLA-A2. For this, 1×10⁵ PBMC derived from healthy, HLA-A2-positive donors were sown, in 100 μl of medium (RPMI 1640, 10% heat-inactivated AB plasma), in each well of a 96-well plate. 20 μg of peptide/ml were added to the culture medium. The cells were incubated at 37° C. and restimulated once per week with irradiated peptide-loaded autologous PBMC. The stimulator PBMC were incubated for 4 hours in medium containing 20 μg of peptide/ml. After the irradiation, they were diluted down to 1×10⁴/100 μl with medium and added to the peptide-stimulated PBMC. For this, half the medium was aspirated from each well and 100 μl of the stimulator PBMC suspension were added. 10 U of IL-2/ml and 10 U of IL-7/ml were added from the 3rd stimulation onwards. From the 5th stimulation onwards, restimulation was carried out in the same manner using peptide-loaded autologous B lymphoblast cell lines (BLCL).

For in vitro vaccination using allogenic tumor cells, HeLa cells were transfected (H7.1-A2) with CD80 and HLA-A2. These cells were irradiated and used for stimulating PBMC at a concentration of 1×10⁴/well. Restimulation was carried out 3 times in an analogous manner to peptide stimulation.

C ELISpot Assay

1×10⁴ stimulator cells (K-A2 cells, that is K-562 cells (ATCC CCL-243) were transfected with an HLA A2 expression vector, PBMC or dendritic cells) were sown, in 60 μl of medium (RPMI 1640, 0.4% human albumin), in one well of a 96-well round-bottomed cell culture plate and loaded overnight with 50 μg of peptide/ml. On the following day, 5×10⁴ T cells, in 60 μl of medium (containing 10 U each of IL-2 and IL-7/ml), were added to each well and the plate was incubated for 4 hours. A Millipore nitrocellulose HA plate was coated overnight, at 4° C., with the anti-human IFN-γ monoclonal antibody 1-Dlk (10 μg/ml in PBS, 60 μl/well, Hölzel Diagnostika, Cologne). On the following day, the plate was washed 3 times for in each case 5 min with 150 μl of sterile PBS per well and then blocked with medium for 1 hour at 37° C. The blocking medium was removed and the cells were transferred from the round-bottomed cell culture plate to the filter plate and then incubated for a further 20 to 22 hours. After that, the cells and the medium were removed and the filter plate was washed 6 times for 2 min with PBS/Tween 20 (0.05%). The antibody Ab-7-B6-1-biotin was then added (2 μg/ml in PBS/BSA, 60 μl/well, Hölzel Diagnostika) and the plate was incubated at 4° C. overnight. On the following day, the filter plate was washed once again 6 times for 2 minutes with PBS/Tween 20 (0.05%) and streptavidin-AP (50 ng/ml in PBS, Sigma (Deisenhofen), 100 μl/well) was added. After a 2-hour incubation at room temperature, the filter plate was washed 3 times for 2 minutes with PBS/Tween 20 (0.05%) and 3 times for 2 minutes with PBS. 60 μl of BCIP/NBT substrate (Sigma, Deisenhofen) were then added and the plate was incubated at room temperature for a further 1 to 2 hours until bluish black spots appeared. The reaction was stopped with water and the plate was dried. The spots were counted using a ZEISS-VISION (Halbergmoos) ELISpot readout system.

D Cytotoxicity Test

The cytotoxicity of HPV18 E7₈₆₋₉₄-specific T cells was determined in a chromium release assay. Autologous BLCL were used as the target cells. In each case, 5×10⁵ cells were taken up in 100 μl of medium (RPMI 1640, 10% FCS) and treated with 20 μl of ⁵¹chromium (NEN). For loading the BLCL with peptide, 50 μg of the corresponding peptide were added/ml. After that, the cells were incubated at room temperature for 2 hours while being carefully resuspended every 20-30 min. The cells were then washed 3 times with 5 ml of medium. For the sedimenting, the cells were in each case centrifuged at 1500 rpm for 5 min and then carefully resuspended. The targets were taken up in medium and adjusted to a density of 1×10⁵ cells/ml. 40 μl of this cell suspension were in each case added to the effectors and CCL-243 K-562 cells which were already sown in the wells of a 96-well pointed-bottom cell culture plate.

The effectors (epitope-specific T cells) were harvested and taken up in medium (RPMI 1640, 10% FCS, 10 U of IL-2 and IL-7/ml). 80 μl of this cell suspension were in each case pipetted (duplicates) into a well in a 96-well pointed bottom cell culture plate. At the same time, the cell count/ml was adjusted so as to ensure that the desired effector/target ratio was obtained. Unlabeled K-562 cells (40 μl, 20-fold excess in relation to the target cells) were added to the effectors in order to completely block any NK cell activity which might possibly be present in the cell lines. The effectors were incubated with the K-562 cells for at least 30 minutes before the labeled target cells were added.

As controls (6-fold assay samples), target cells were, on the one hand, incubated with K-562 and medium (low-release control) and, on the other hand, incubated with k562, medium and Tween 20 (high-release control). In order to check whether the NK activity was completely blocked, effectors were also incubated with unlabeled and labeled K-562.

The assay samples were incubated for 4 hours at 37° C., 98% atmospheric humidity and 5% CO₂. After that, the culture plates were centrifuged at 700 rpm for 5 min in order to sediment the cells. 100 μl of the supernatant from each well was then pipetted, without whirling up the cell pellet, onto an Opti-Plate scintillation tabletting plate and left to stand overnight at room temperature. On the following day, 150 μl of Microscint 40 scintillation fluid (Canberra-Packard, Deieich) were added and the numbers of distintegrations per minute in the individual assay samples were measured in the scintillation counter (Topcount, Canberra-Packard, Dreieich). In conclusion, the mean values and standard deviations of the multiple assay samples were computed.

The specific lysis was calculated using the following formula:

specific lysis (%)=100×(result−low release)×(high release−low release)⁻¹

E Culturing Tumor-infiltrating Lymphocytes (TIL)

TIL were caused to grow from the biopsies of tumors present in female patients exhibiting HPV18 and HLA-A2 positivity by culturing the TIL in AIM V medium (Gibco-Invitrogen, Karlsruhe) containing in each case 100 U of IL-2 and IL-7/ml and 0.125 μl of Dynal beads T cell expander (Dynal, Hamburg)/well. The specificity of these TIL for the HPV18 E7₈₆₋₉₄ peptide epitope was examined directly in an ELISpot assay.

F Results

In order to determine which HLA-A2-restricted peptides of the HPV18 E7 protein are processed and presented by tumor cells, a T cell line was generated by means of in vitro vaccination using HeLa CD80/HLA-A2 transfectants (H7.1-A2). A specific reaction against the predicted synthetic peptide FQQLFLNTL was seen in the IFN-γ ELISpot analysis (see upper part of FIG. 17). As compared with other predicted peptides, this peptide has a relatively low binding affinity for HLA-A2. Despite this, specific T cells were detected. HPV16 E7₂₈₋₃₆, which did not induce any IFN-γ secretion, was used as the control peptide. This suggests that this peptide is presented by H7.1-A2 cells.

In order to test whether HPV18 E7₈₆₋₉₄-specific T cells possess cytolytic activity, T cell lines were prepared against the synthetic peptide. The T cells were tested in the chromium release assay. Autologous peptide-loaded BLCL were used as the target cells. A specific lysis of approx. 20% was observed at an effector to target ratio of 30:1 (see lower part of FIG. 17). No specific lysis was measured against the control peptides HPV18 E7₇₋₁₅ and HPV16 E6₂₈₋₃₆.

In order to check whether the HPV18 E7₈₆₋₉₄ peptide is also naturally processed by the immunoproteasome, T cell lines were prepared by stimulating with antigen-loaded autologous dendritic cells (DC). The DC were loaded with recombinant HPV18 E7 protein or a pool composed of overlapping 20 mer peptides representing the entire HPV18 E7 (see upper part of FIG. 18: “E7 protein-induced” and “E7 peptide pool-induced”, respectively). The specificity of the induced T cells for the HPV18 E7₈₆₋₉₄ epitope was tested in an ELISpot assay. HPV18 E7₈₆₋₉₄-specific T cells were detected in both T cell lines (see upper part of FIG. 18). This means that the immunoproteasome processes the epitope.

The biological relevance of the HPV18 E7₈₆₋₉₄ epitope was investigated using TIL populations. TIL were isolated from a tumor biopsy obtained from an HPV18- and HLA-A2-positive female patient and linearly expanded in vitro without any antigen-specific restimulation. In the ELISpot assay, they were confronted with HPV18 E7₈₆₋₉₄-loaded K-A2 (HLA-A2-transfected K-562 cells as stimulators).

While there is a marked reaction against HPV18 E7₈₆₋₉₄ in the TIL populations, there is no such reaction against the HPV18 E7₇₋₁₅ epitope described by Rudolf MP et al. (2001, Clinical Cancer Research 7 (3 Suppl) :pp788-795) (see lower part of FIG. 18). The peptide HPV18 E7₇₋₁₅ has a binding affinity for HLA-A2 which is 2.5 times higher. These results point to HPV18 E7₈₆₋₉₄-specific T cells being naturally present in tumors, something which underlines the relevance of the peptide epitope which has been found.

### 19. Summary of Examples 3 to 18

The data obtained in examples 3 to 18 are summarized in tables 3 and 4:

TABLE 3
HPV16 peptides/epitopes

| | | Rel. | | Haplo- | Epitope | |
|---|---|---|---|---|---|---|
| Name | Sequence | position | Species | type | type | Data in |
| P2 | YLPPVPVSKVVSTDEYVART | L1 12-31 | human | | T_{C} | Example 9 |
| P3 | STDEYVARTNIYYHAGTSRL | L1 23-42 | murine | H2b | | Example 2 |
| P3 | STDEYVARTNIYYHAGTSRL | L1 23-42 | human | | T_{C} | Examples |
| | | | | | | 9, 13 |
| P5 | VGHPYFPIKKPNNNKILVPK | L1 45-64 | human | | T_{C} | Example 13 |
| P7 | GLQYRVFRIHLPDPNKFGFP | L1 67-86 | human | | T_{C} | Example 9 |
| P10 | WACVGVEVGRGQPLGVGISG | L1 100-119 | human | | T_{H} | Example 6 |
| P11 | QPLGVGISGHPLLNKLDDTE | L1 111-130 | human | | T_{H} | Example 6 |
| P15 | QLCLIGCKPPTGEHWGKGSP | L1 155-174 | human | | T_{H} | Example 6 |
| P18 | LELINTVIQDGDMVDTGFGA | L1 188-207 | murine | H2b | | Example 2 |
| P18 | LELINTVIQDGDMVDTGFGA | L1 188-207 | human | | T_{H} | Example 6 |
| P19 | DMVDTGFGAMDFTTLQANKS | L1 199-218 | murine | H2b | | Example 2 |
| P31 | VTVVDTTRSTNNSLCAAIST | L1 331-350 | human | | T_{H} | Example 6 |
| P33 | TTYKNTNFKEYLRHGEEYDL | L1 353-372 | murine | H2b | | Example 3 |
| P33 | TTYKNTNFKEYLRHGEEYDL | L1 353-372 | human | | T_{C} | Example 10 |
| P35 | IFQLCKITLTADVMTYIHSM | L1 375-394 | murine | H2b | | Example 2 |
| P38 | PPPGGTLEDTYRFVTSQAIA | L1 408-427 | human | | T_{H2} | Example 8 |
| P39 | RFVTSQAIACQKHTPPAPKE | L1 419-438 | human | | T_{H1}, T_{H2} | Example 8 |
| P41 | LKKYTFWEVNLKEKFSADLD | L1 441-460 | human | | T_{C} | Example 13 |
| P43 | PLGRKFLLQAGMHGDTPTLH | L1 463-482 | murine | H2b | | Example 2 |
| P46 | YCYEQLNDSSEEEDEIDGPA | E7 23-42 | human | | T_{C} | Example 13 |

TABLE 4
HPV18 peptides/epitopes

| | | Rel. | | Haplo- | Epitope | | |
|---|---|---|---|---|---|---|---|
| Name | Sequence | position | Species | type | type | Data in | |
| Q3 | NTDDYVTRTSIFYHAGSSRL | L1 23-42 | murine | H2b | T_{C} | Example 16 | |
| Q4 | FYHAGSSRLLTVGNPYFRVP | L1 34-53 | murine | H2b | T_{C} | Example 16 | |
| Q5 | VGNPYFRVPAGGGNKQDIPK | L1 45-64 | murine | H2k | | Example 4 | |
| Q6 | GGNKQDIPKVSAYQYRVFRV | L1 56-75 | human | | T_{H} | Example 5 | |
| Q9 | SIYNPETQRLVWACAGVEIG | L1 89-108 | human | A24 | T_{C} | Example 7 | |
| | respectively | 90-98 | | | | | |
| | IYNPETQRL | | | | | | |
| Q22 | PDYLOMSADPYGDSMFFCLR | L1 232-251 | murine | H2k | | Example 4 | |
| Q23 | GDSMFFCLRREQLFARHFWN | L1 243-262 | murine | H2k | | Example 4 | |
| Q30 | NNGVCWHNQLFVTVVDTTRS | L1 320-339 | human | | T_{H1} | Example 14 | |
| Q38 | PPPPTTSLVDTYRFVQSVAI | L1 408-427 | human | | T_{H1}, T_{H2}, | Examples | |
| | | | | | T_{C} | 11, 12 | |
| Q39 | YRFVQSVAITCQKDAAPAEN | L1 419-438 | human | | | T_{H1}, T_{H2}, | Examples |
| | | | | | T_{C} | 11, 12 | |
| Q41 | PYDKLKFWNVDLKEKFSLDL | L1 441-460 | murine | H2k | | Example 4 | |
| Q43 | YPLGRKFLVQAGMHGPKATL | L1 463-482 | murine | H2k | | Example 4 | |
| Q43 | YPLGRKFLVQAGMHGPKATL | L1 463-474 | murine | H2b | | Example 17 | |
| | | E7 1-8 | | | | | |
| Q44 | MHGPKATLQDIVLHLEPQNE | E7 1-20 | murine | H2b | T_{C} | Example 17 | |
| Q44 | MHGPKATLQDIVLHLEPQNE | E7 1-20 | murine | H2k | | Example 4 | |
| Q46 | VDLLCHEQLSDSEEENDEID | E7 23-42 | human | | T_{H1} | Examples | |
| | | | | | | 11, 12 | |
| Q47 | SEEENDEIDGVNHQHLPARR | E7 34-53 | human | | T_{H1}, T_{C} | Examples | |
| | | | | | | 11, 12 | |
| Q49 | PQRHTMLCMCCKCEARIKLV | E7 56-75 | murine | H2k | T_{C} | Example 17 | |
| Q51 | SSADDLRAFQQLFLNTLSFV | E7 78-97 | murine | H2k | | Example 4 | |
| L1₄₇₄ | GMHGPKATL | L1 474 | murine | H2b | T_{C} | Example 16 | |
| E7₁₋₈ | | E7 1-8 | | | | | |
| E7₂₋₁₁ | HGPKATLQDI | E7 2-11 | murine | H2b | T_{C} | Example 16 | |
| E7₁₋₈ | MHGPKATL | E7 1-8 | murine | H2b | T_{C} | Example 16 | |
| E7₈₆₋₉₄ | FQQLFLNTL | E7 86-94 | human | HLA-A2 | T_{C} | Example 18 | |

1. A T cell epitope having an amino acid sequence
YLPPVPVSKVVSTDEYVART, STDEYVARTNIYYHAGTSRL,
VGHPYFPIKKPNNNKILVPK, GLQYRVFRIHLPDPNKFGFP,
WACVGVEVGRGQPLGVGISG, QPLGVGISGHPLLNKLDDTE,
QLCLIGCKPPIGEHWGKGSP, LELINTVIQDGDMVDTGFGA,
DMVDTGFGAMDFTTLQANKS, VTVVDTTRSTNMSLCAAIST,
TTYKNTNFKEYLRHGEEYDL, IFQLCKITLTADVMTYIHSM,
PPPGGTLEDTYRFVTSQAIA, RFVTSQAIACQKHTPPAPKE,
LKKYTFWEVNLKEKFSADLD, PLGRKFLLQAGMHGDTPTLH,
YCYEQLNDSSEEEDEIDGPA, VGNPYFRVPAGGGNKQDIPK,
GGNKQDIPKVSAYQYRVFRV, SIYNPETQRLVWACAGVETG,
IYNPETQRL, PDYLQMSADPYGDSMFFCLR,
GDSMFFCLRREQLFARHFWN, NNGVCWHNQLFVTVVDTTRS,
PPPPTTSLVDTYRFVQSVAI, YRFVQSVAITCQKDAAPAEN,
PYDKLKFWNVDLKEKFSLDL, YPLGRKFLVQAGMHGPKATL,
MHGPKATLQDIVLHLEPQNE, VDLLCHEQLSDSEEENDEID,
SEEENDEIDGVNHQHLPARR, SSADDLRAFQQLFLNTLSFV,
NTDDYVTRTSIFYHAGSSRL, FYHAGSSRLLTVGNPYFRVP,
PQRHTMLCMCCKCEARIKLV, GMHGPKATL, HGPKATLQDI,
MHGPKATL, or FQQLFLNTL
and/or a functionally active variant thereof.

2. The T cell epitope as claimed in claim 1, characterized in that said variant possesses a sequence homology with
YLPPVPVSKVVSTDEYVART, STDEYVARTNIYYHAGTSRL,
VGHPYFPIKKPNNNKILVPK, GLQYRVFRIHLPDPNKFGFP,
NACVGVEVGRGQPLGVGISG, QPLGVGISGHPLLNKLDDTE,
QLCLIGCKPPIGEHWGKGSP, LELINTVIQDGDMVDTGFGA,
DMVDTGFGAMDFTTLQANKS, VTVVDTTRSTNMSLCAAIST,
TTYKNTNFKEYLRHGEEYDL, IFQLCKITLTADVMTYIHSM,
PPPGGTLEDTYRFVTSQAIA, RFVTSQAIACQKHTPPAPKE,
LKKYTFWEVNLKEKFSADLD, PLGRKFLLQAGMHGDTPTLH,
YCYEQLNDSSEEEDEIDGPA, VGNPYFRVPAGGGNKQDIPK,
GGNKQDIPKVSAYQYRVFRV, SIYNPETQRLVWACAGVEIG,
IYNPETQRL, PDYLQMSADPYGDSMFFCLR,
GDSMFFCLRREQLFARHFWN, NNGVCWHNQLFVTVVDTTRS,
PPPPTTSLVDTYRFVQSVAI, YRFVQSVAITCQKDAAPAEN,
PYDKLKFWNVDLKEKFSLDL, YPLGRKFLVQAGMHGPKATL,
MHGPKATLQDIVLHLEPQNE, VDLLCHEQLSDSEEENDEID,
SEEENDEIDGVNHQHLPARR, SSADDLRAFQQLFLNTLSFV,
NTDDYVTRTSIFYHAGSSRL, FYHAGSSRLLTVGNPYFRVP,
PQRHTMLCMCCKCEARIKLV, GMHGPKATL, HGPKATLQDI,
MHGPKATL, or FQQLFLHTL
of at least approx. 65%, preferably at least approx. 75% and in particular at least approx. 85% at the amino acid level.

3. The T cell epitope as claimed in claim 1, characterized in that said variant has structural homology with
YLPPVPVSKVVSTDEYVART, STDEYVARTNIYYHAGTSRL,
VGHPYFPIKKPNNNKILVPK, GLQYRVFRIHLPDPNKFGFP,
WACVGVEVGRGQPLGVGISG, QPLGVGISGHPLLNKLDDTE,
QLCLIGCKPPIGEHWGKGSP, LELINTVIQDGDMVDTGFGA,
DMVDTGFGAMDFTTLQANKS, VTVVDTTRSTNMSLCAAIST,
TTYKNTNFKBYLRHGEEYDL, IFQLCKITLTADVMTYIHSM,
PPPGGTLEDTYRFVTSQAIA, RFVTSQAIACQKHTPPAPKE,
LKKYTFWEVNLKEKFSADLD, PLGRKFLLQAGMHGDTPTLH,
YCYEQLNDSSEEEDEIDGPA, VGNPYFRVPAGGGNKQDIPK,
GGNKQDIPKVSAYQYRVFRV, SIYNPETQRLVWACAGVEIG,
IYNPETQRL, PDYLQMSADPYGDSMFFCLR,
GDSMFFCLRREQLFARHFWN, NNGVCWHNQLFVTVVDTTRS,
PPPPTTSLVDTYRFVQSVAI, YRFVQSVAITCQKDAAPAEN,
PYDKLKFWNVDLKEKFSLDL, YPLGRKFLVQAGMHGPKATL,
MHGPKATLQDIVLHLEPQNE, VDLLCHEQLSDSEEENDEID,
SEEENDEIDGVNHQHLPARR, SSADDLRAFQQLFLNTLSFV,
NTDDYVTRTSIFYHAGSSRL, FYHAGSSRLLTVGNPYFRVP,
PQRHTMLCMCCKCEARIKLV, GMHGPKATL, HGPKATLQDI,
MHGPKATL, or FQQLFLHTL

4. The T cell epitope as claimed in one of claims 1-3, characterized in that the T cell epitope induces a cytotoxic response or mediates a T helper cell function.

5. A compound containing a T cell epitope as claimed in one of claims 1 to 4, with the compound not being any naturally occurring L1 protein derived from a papillomavirus and, in the case of an HPV-16 T cell epitope, not being any exclusively N-terminal or exclusively C-terminal deletion mutant of a naturally occurring L1 protein derived from a papillomavirus.

6. The compound as claimed in claim 5, characterized in that the compound is a polypeptide, in particular a fusion protein.

7. The compound as claimed in claim 5 or 6, characterized in that the compound is a polypeptide of at least approx. 50 amino acids, preferably of at least approx. 35 amino acids, in particular of at least approx. 20 amino acids, and, in a particularly preferred manner, of at least approx. 9-13 amino acids, in length.

8. The compound as claimed in one of claims 5-7, characterized in that the compound contains a chemical, radioactive, nonradioactive isotopic and/or fluorescent labeling of the T cell epitope and/or of said fusion protein, and/or a chemical modification of the T cell epitope and/or fusion protein.

9. A nucleic acid, characterized in that it encodes a T cell epitope as claimed in one of claims 1-4 or a compound containing a T cell epitope as claimed in one of claims 5-8.

10. A vector, in particular an expression vector, characterized in that it contains a nucleic acid as claimed in claim 9.

11. A cell, characterized in that it contains, preferably presents, at least one T cell epitope as claimed in one of claims 1-4 or a compound as claimed in one of claims 5-8.

12. The cell as claimed in claim 11, characterized in that the cell is transfected, transformed and/or infected with a nucleic acid as claimed in claim 9 and/or a vector as claimed in claim 10.

13. The cell as claimed in claim 11, characterized in that the cell was incubated with at least one T cell epitope as claimed in one of claims 1-4, at least one compound as claimed in one of claims 5-8 and/or at least one complex as claimed in one of claims 15-17 containing a T cell epitope as claimed in one of claims 5-8.

14. The cell as claimed in claim 11 or 12, characterized in that the cell is a B cell, a macrophage, a dendritic cell or a fibroblast, in particular a JY cell, T2 cell, CaSki cell or EBV-transformed cell.

15. A complex comprising a T cell epitope as claimed in one of claims 1-4 or a compound as claimed in one of claims 5-8 and at least one further compound.

16. The complex as claimed in claim 15, characterized in that the complex contains at least one MHC class I molecule, preferably as HLA A2.01, A1 or A24 tetramer.

17. The complex as claimed in claim 16, characterized in that said MHC class I molecule is a human MHC class I molecule, in particular an HLA A2.01, A1 or A24 molecule.

18. A method for the in-vitro detection of the activation of T cells by at least one T cell epitope as claimed in one of claims 1-4 or by at least one compound containing a T cell epitope as claimed in one of claims 1-4, which contains the following steps:
a) stimulating cells with at least one said compound;
b) adding at least one target cell, which is presenting a T cell epitope as claimed in one of claims 1-4, or a complex as claimed in one of claims 15-17, and
c) determining the activation of T cells.

19. The method as claimed in claim 18, characterized in that, after step a), it contains the following additional step a′):
a′) coculturing cells for at least approx. 1 week, in particular at least approx. 8 weeks, with:
(i) at least one target cell which is loaded with a T cell epitope as claimed in one of claims 1-4, with a compound as claimed in one of claims 5-8, at least one complex as claimed in one of claims 15-17, at least one capsomere, at least one stable capsomere, at least one VLP, at least one CVLP and/or at least one virus,
(ii) at least one complex as claimed in one of claims 15-17,
(iii) and/or at least one target cell which is presenting a T cell epitope as claimed in one of claims 1-4,
before step b) follows.

20. The method for preparing a target cell as claimed in one of claims 11, 13, 14, 18 or 19, characterized in that the target cell is incubated with at least one T cell epitope as claimed in one of claims 1-4, with at least one compound as claimed in one of claims 5-8 and/or at least one complex as claimed in one of claims 15-17 containing a T cell epitope as claimed in one of claims 5-8.

21. The method for preparing a target cell as claimed in one of claims 11, 12, 14, 18 or 19, characterized in that the target cells is transfected, transformed and/or infected with a nucleic acid as claimed in claim 9 and/or a vector as claimed in claim 10.

22. The method for preparing a target cell as claimed in claim 20 or 21, characterized in that the target cell is a B cell, a macrophage, a dendritic cell or a fibroblast, in particular a JY cell, T2 cell, CaSki cell or EBV-transformed cell.

23. The method as claimed in claim 18 or 19, characterized in that the following step a″) is carried out in place of step a):
a″) isolating and preparing samples containing T cells and then culturing them.

24. A test system for the in-vitro detection of the activation of T cells comprising:
a) at least one T cell epitope as claimed in one of claims 1-4, at least one compound as claimed in one of claims 5-8, at least one vector as claimed in claim 10, at least one cell as claimed in one of claims 11-14 and/or at least one complex as claimed in one of claims 15-17, and
b) immune system effector cells, preferably T cells, in particular cytotoxic T cells or T helper cells.

25. The use of at least one T cell epitope as claimed in one of claims 1-4, of at least one compound as claimed in one of claims 5-8, of at least one vector as claimed in claim 10, of at least one cell as claimed in one of claims 11-14, and/or of at least one complex as claimed in one of claims 15-17, for inducing or for detecting an immune response.

26. A pharmaceutical or diagnostic agent comprising at least one T cell epitope as claimed in one of claims 1-4, at least one compound as claimed in one of claims 5-8, at least one vector as claimed in claim 10, at least one cell as claimed in one of claims 11-14, and/or at least one complex as claimed in one of claims 15-17 and, where appropriate, a pharmaceutically acceptable carrier.

27. A pharmaceutical or diagnostic agent as claimed in claim 26, characterized in that at least one T cell epitope as claimed in one of claims 1-4, at least one compound as claimed in one of claims 5-8, at least one vector as claimed in claim 10, at least one cell as claimed in any one of claims 11-14, and/or at least one complex as claimed in one of claims 15-17 is present in solution, is bound to a solid matrix and/or is treated with an adjuvant.
